(19) United States
(12) Patent Application Publication
(10) Pub. No.: US 2025/0111951 A1
(43) Pub. Date: Apr. 3, 2025

(54) METHODS FOR DESCRIBING VAGINAL MICROBIOMES AND USE OF THE SAME IN PROGNOSTIC, DIAGNOSTIC, AND THERAPEUTIC STRATEGIES FOR BACTERIAL VAGINOSIS

(71) Applicant: UNIVERSITY OF MARYLAND, BALTIMORE, Baltimore, MD (US)

(72) Inventors: Johanna B. HOLM, Baltimore, MD (US); Bing MA, Clarksville, MD (US); Jacques RAVEL, Laurel, MD (US); Pawel GAJER, Fort Myers, FL (US)

(73) Assignee: UNIVERSITY OF MARYLAND, BALTIMORE, Baltimore, MD (US)

(21) Appl. No.: 18/904,823

(22) Filed: Oct. 2, 2024

Related U.S. Application Data
(60) Provisional application No. 63/541,969, filed on Oct. 2, 2023.

(51) Int. Cl. G16H 50/30; C12Q 1/04; G16H 10/60; G16H 50/70

(52) U.S. Cl. CPC G16H 50/30; C12Q 1/04; G16H 10/60; G16H 50/70

(57) ABSTRACT

A Lactobacillus-dominated vaginal microbiome provides the first line of defense against numerous adverse genital tract health outcomes. However, there is limited understanding of the mechanisms by which the vaginal microbiome modulates protection. Metagenomic community state types (mgCSTs), which uses metagenomic sequences to describe and define vaginal microbiomes based on both composition and function are described herein, along with their use in prognostic, diagnostic and therapeutic applications against bacterial vaginosis.

### STATEMENT OF FEDERALLY SPONSORED RESEARCH AND DEVELOPMENT

This invention was made with government support under Grant Numbers AI163413, AI136400, AI084044, AI083264, AI116799 and NR015495 awarded by National Institutes of Health. The government has certain rights in the invention.

### BACKGROUND OF INVENTION

The vaginal microbiome plays a vital role in gynecological and reproductive health. Lactobacillus-predominated vaginal microbiota constitute the first line of defense against infection. Protective mechanisms include lactic acid production by Lactobacillus spp., which acidifies the vaginal microenvironment and elicits anti-inflammatory effects [1-4]. This environment wards off non-indigenous organisms, including causative agents of sexually transmitted infections (STIs) such as HIV, and bacteria associated with bacterial vaginosis (BV) [5-7]. However, vaginal Lactobacillus spp. are functionally diverse. For example, L. crispatus and L. gasseri are capable of producing both the D- and L-isomers of lactic acid, L. jensenii produces only the D-isomer, and L. iners only the L-isomer [4, 8]. These key features have implications for susceptibilities to pathogens [9, 10].

The vaginal microbiota has been previously shown to cluster into community state types (CSTs) that reflect differences in bacterial species composition and abundance [1, 11]. Lactobacillus spp. predominate four of the five CSTs (CST I: L. crispatus; CST II: L. gasseri; CST III: L. iners; CST V: L. jensenii). In contrast, CST IV communities are characterized by a paucity of lactobacilli and the presence of a diverse array of anaerobes such as Gardnerella vaginalis and “Ca. Lachnocurva vaginae”. CST IV is found, albeit not exclusively, during episodes of BV, a condition associated with increased risk to sexually transmitted infections, including HIV, as well as preterm birth and other gynecological and obstetric adverse outcomes [12-20].

BV is characterized by a lack of Lactobacillus spp. in the vagina and the presence of a diverse, anaerobic microbiota. It is a common form of vaginitis affecting women of all ages, races and socioeconomic statuses with an estimated prevalence of 23-29% [43,57] and closer to 50% in sub-Saharan Africa [78-81]. In the U.S., women of African or Latin descent experience higher rates of prevalence (33.2% and 30.7%, respectively) compared to white or Asian women [57]. BV is clinically defined by observing 3 of 4 Amsel's criteria (Amsel-BV; vaginal pH >4.5; abnormal discharge; and on wet mount, presence of clue cells and fishy odor with 10% KOH) [21]. Patients presenting with symptoms and satisfying the Amsel's criteria (symptomatic Amsel-BV) are treated with antibiotics, however, efficacy is poor, and recurrence is common [21-24]. In research settings, BV is often defined by scoring Gram stained vaginal smears (Nugent-BV) [25] or molecular typing of bacterial composition by sequencing marker genes (molecular-BV) [26]. There is no definition of BV that relies on both the composition and function of the microbiome.

Species-level composition of the vaginal microbiota used for CSTs may not suffice to accurately capture associations between the vaginal microbiome and outcomes of interest because functional differences exist between strains of the same species. For example, in the skin microbiome strains of Staphylococcus aureus or Streptococcus pyogenes elicit different acute immune responses [27]. Similarly, genomic and functional analyses of Lactobacillus rhamnosus strains demonstrate distinct adaptations to specific niches (for example, the gut versus the oral cavity) [28]. While functional differences likely exist between strains of the same species in the vaginal microbiota, metagenomic studies show that combinations of multiple strains co-exist within a single vaginal microbiome [29, 30]. These strain assemblages are known as metagenomic subspecies or mgSs [29], and are important to consider as they potentially impact the functional diversity and resilience of a species in a microbiome.

Determining the mechanistic consequences and health outcomes associated with mgSs may improve precision of risk estimates and interventions.

### BRIEF SUMMARY OF INVENTION

Relevant to the characterization of metagenomic subspecies (mgSs), recent findings have shown that multiple strains of the same species are commonly observed in the vaginal microbiome [29], and that samples can be clustered into mgSs defined by unique strain combinations represented by species-specific gene sets, and thus unique sets of functions. These critical observations led to the conceptualization of vaginal microbiome classifications based on their mgSs compositions and abundance, and thus defined by both species' composition and functions, i.e., metagenomic community state types (MgCSTs). MgCSTs describe vaginal microbiomes through a new lens, one that includes both compositional and functional dimensions.

MgCSTs are composed of unique combinations of mgSs. A two-step classifier that assigns metagenomic subspecies and mgCSTs was developed and validated, and it is designed to work in concert with the vaginal non-redundant gene database, VIRGO [29]. This classifier will facilitate reproducibility and comparisons across studies.

MgCSTs allow integration of the taxonomic composition and functional potential of vaginal microbiomes in prognostic, diagnostic and therapeutic strategies, as reported herein. The present invention takes advantage of MgCST classifications for use in prognostic, diagnostic and therapeutic strategies associated with bacterial vaginosis (BV), along with other important uses.

Thus, and in non-limiting examples, the invention is drawn to methods of characterizing a vaginal microbiome, methods of identifying a subject predisposed to develop bacterial vaginosis, methods of identifying a subject predisposed to re-develop bacterial vaginosis, methods of diagnosing bacterial vaginosis in a subject, methods of treating bacterial vaginosis in a subject, and methods of preventing bacterial vaginosis in a subject.

In particular, and in a first embodiment, the invention is directed to methods of characterizing a vaginal microbiome, comprising obtaining a vaginal microbiome sample from a subject, determining the metagenomic community state type (MgCST) of the sample, and classifying the MgCST as one of MgCSTs 1-27.

In a second embodiment, the invention is directed to methods of identifying a subject predisposed to develop bacterial vaginosis, comprising obtaining a vaginal microbiome sample from a subject, and determining the metagenomic community state type (MgCST) of the sample, wherein when the MgCST of the subject is determined to be a MgCST associated with bacterial vaginosis, the subject is identified as a subject predisposed to develop bacterial vaginosis.

In certain aspects of this embodiment, a MgCST associated with bacterial vaginosis is one or more of MgCSTs 12 and 17-25.

In a third embodiment, the invention is directed to methods of identifying a subject predisposed to re-develop bacterial vaginosis, comprising determining the metagenomic community state type (MgCST) of a vaginal microbiome of a subject after treatment for bacterial vaginosis and classifying it as one of MgCSTs 1-27, wherein when the MgCST of the subject is classified as one or more of MgCSTs 12 and 17-25, the subject is predisposed to re-develop bacterial vaginosis.

In certain aspects of this embodiment, when the MgCST of the subject is classified as one or more of MgCSTs 19 and 22, the subject is predisposed to re-develop bacterial vaginosis.

In a fourth embodiment, the invention is directed to methods of diagnosing bacterial vaginosis in a subject, comprising obtaining a vaginal microbiome sample from a subject, and determining the metagenomic community state type (MgCST) of the sample, wherein when the MgCST of the subject is determined to be a MgCST associated with bacterial vaginosis, the subject is diagnosed as having bacterial vaginosis.

In certain aspects of this embodiment, a MgCST associated with bacterial vaginosis is one or more of MgCSTs 12 and 17-25.

In a fifth embodiment, the invention is directed to methods of treating bacterial vaginosis in a subject, comprising obtaining a vaginal microbiome sample from a subject, determining the metagenomic community state type (MgCST) of the sample, and administering a therapeutically effective amount of an antibacterial agent to the subject when the MgCST of the subject is determined to be a MgCST associated with bacterial vaginosis.

In certain aspects of this embodiment, a MgCST associated with bacterial vaginosis is one or more of MgCSTs 12 and 17-25.

In certain aspects of this embodiment, the antibacterial agent is metronidazole or clindamycin.

In a sixth embodiment, the invention is directed to methods of preventing bacterial vaginosis in a subject, comprising obtaining a vaginal microbiome sample from a subject, determining the metagenomic community state type (MgCST) of the sample, and administering a prophylactically effective amount of an antibacterial agent to the subject when the MgCST of the subject is determined to be a MgCST associated with bacterial vaginosis.

In certain aspects of this embodiment, a MgCST associated with bacterial vaginosis is one or more of MgCSTs 12 and 17-25.

In certain aspects of this embodiment, the antibacterial agent is metronidazole or clindamycin.

In each of the relevant embodiments and aspects of the invention as summarized herein, the methods of the invention may further comprise determining one or more of (a) age of the subject, (b) race of the subject, (c) vaginal pH of the subject, and (d) Gram stain assessment of the subject.

In each of the relevant embodiments and aspects of the invention as summarized herein, the methods of the invention may further comprise determining one or more metagenomic subspecies (mgSs) cluster in the sample.

In each of the relevant embodiments and aspects of the invention as summarized herein, the MgCST may be determined by a two-step classifier that assigns one or both of MgSs and mgCSTs. The classifier may be based on mgS composition or abundance, or both. The classifier may use a vaginal non-redundant gene database (VIRGO).

In each of the relevant embodiments and aspects of the invention as summarized herein, the subject may be a human.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits of the invention.

### BRIEF DESCRIPTION OF DRAWINGS

The patent or application file contains at least one drawing executed in color. Copies of this patent or patent application publication with color drawing(s) will be provided by the Office upon request and payment of the necessary fee.

FIG. 1. Vaginal Metagenomic Community State Types (mgCSTs). Using 1,890 metagenomic samples, 27 mgCSTs were identified: mgCSTs 1-16 are predominated by metagenomic subspecies of Lactobacillus spp., mgCSTs 17-19 by metagenomic subspecies of “Ca. Lachnocurva vaginae”, mgCSTs 20-25 by metagenomic subspecies of the genus Gardnerella, and mgCST 27 contains samples without a predominant metagenomic subspecies.

FIG. 2. MgCSTs are associated with self-reported race ((a), n=1,441) and age categories ((b), n=1,623) Within-mgCST distributions are compared to study-wide distribution (*:p<0.05, **:p<0.01, ***:p<0.001). (c) The distribution of mgCSTs differs by race. Vaginal microbiomes from black women have the smallest proportion of L. crispatus mgCSTs. “Ca. Lachnocurva vaginae” mgCSTs 17-19 are absent from Asian vaginal microbiomes in this study, as are L. iners mgCST 10 and 14.

FIG. 3. MgCSTs are associated with Nugent Scores ((a), n=968) and vaginal pH categories ((b), n=979). Within-mgCST distributions were compared to study-wide distributions (*:p<0.05, **:p<0.01, ***:p<0.001).

FIG. 4. Clinically diagnosed Amsel bacterial vaginosis (a) and symptomatic Amsel bacterial vaginosis (b) associate with mgCSTs. (a) Within each mgCST, the total number of clinical evaluations per mgCST are indicated. The black bars indicate the proportion of negative Amsel-BV diagnoses, and the colored bars indicate the positive Amsel-BV diagnoses. Within-mgCST proportions were statistically compared to the study-wide proportions of Amsel-BV diagnoses. (b) Of positive Amsel-BV diagnoses in a), the proportions of asymptomatic (light gray) and symptomatic (dark gray) Amsel-BV diagnoses are shown within each mgCST. Within-mgCST proportions were statistically compared to the study-wide proportions of asymptomatic to symptomatic Amsel-BV diagnoses. (*:p<0.05, **:p<0.01, ***:p<0.001).

FIG. 5. The presence of D-lactate dehydrogenase orthologs differ by mgCST. (a) D-lactate dehydrogenase orthologs in VIRGO compared to functionally validated reference, P30901.2. (b) MgCST 2 contains fewer estimated strains of L. crispatus. (c) On average, vaginal pH is higher in mgCST 2. (d) Shannon's H is higher in mgCST 2 than mgCST 1 or 3. c) Microbiome stability is lower in mgCST 2.

FIG. 6. The total number of clinical evaluations per L. iners mgSs are indicated in (a). The black bars indicate the proportion of negative Amsel-BV diagnoses, and the colored bars indicate the positive Amsel-BV diagnoses. (b) Gene presence map represents gene content of L. iners mgSs (columns) and L. iners gene clusters (rows). (c) The total number of clinical evaluations per L. iners gene cluster are indicated. The black bars indicate the proportion of negative Amsel-BV diagnoses, and the colored bars indicate the positive Amsel-BV diagnoses. Within-gene group proportions were statistically compared to the study-wide proportions of Amsel-BV for any samples containing a L. iners gene cluster. (*:p<0.05, **:p<0.01, ***:p<0.001).

FIG. 7. The proportions of Gardnerella genomospecies in a sample differs by Gardnerella mgSs (a). For samples with Amsel-BV evaluations, Amsel-BV status is indicated below each sample (column). (b) Gene clusters of Gardnerella contain genes attributed to a variety of Gardnerella genomospecies (as indicated by colored bars, black bars indicate unknown genomospecies). (c) The total number of clinical evaluations per G. vaginalis gene cluster are indicated. The dark gray bars indicate the proportion of negative Amsel-BV diagnoses, and the colored bars indicate the positive Amsel-BV diagnoses. Within-gene group proportions were statistically compared to the study-wide proportions of Amsel-BV for any samples containing a G. vaginalis gene cluster. (*:p<0.05, **:p<0.01, ***:p<0.001).

FIG. 8. Three external metagenomic datasets were processed with VIRGO and assigned mgCSTs using the mgCST classifier. (a) Most samples were statistically similar to the reference centroid of the assigned mgCST. (b) Samples in each dataset were distributed across mgCSTs. In all cases, the lowest similarity scores were observed in mgCST 27.

FIG. 9A. Case and control definitions for a retrospective case-control analysis within the Longitudinal Study of Vaginal Flora (LSVF).

FIG. 9B. Study design for H1. Identifying mgCSTs associated with BV persistence using archived cervicovaginal lavages from the Longitudinal Study of Vaginal Flora (LSVF).

FIG. 9C. Study design for H2 and H3.

FIG. 10. (A) BV recurrence (rBV) is associated with non-Lactobacillus vaginal microbiota (filled circles, solid confidence intervals, CIs). (B) MgCSTs refine the estimates of risk associated with rBV. The odds of recurrence do not statistically differ if metronidazole is given for the index BV event (unfilled circles, dashed CIs). (C) Bacterial species associated with BV recurrence (red) and resolution (black).

FIG. 11A. Predicted amounts of stain-sharing using output from InStrain.

FIG. 11B. Using already-generated metagenomes from a participant in the LSVF cohort, InStrain iodentified the same P. amnii strains between samples 3-months apart, and these strains were distinct from the index event.

FIG. 12. The vaginal microbiome is associated with risk of incident STIs. mgCSTs 11, 17, 20, 22, and 23, were significantly associated with incident STI compared to the L. crispatus dominated vaginal metagenomic community state type (mgCST) 1. (n=708). (*p<0.05, **p<0.01)

### DETAILED DESCRIPTION OF THE INVENTION

### I. DEFINITIONS

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

### II. THE PRESENT INVENTION

MgCSTs are categories of microbiomes classified using taxonomy and the functional potential encoded in their metagenomes. MgCSTs reflect unique combinations of metagenomic subspecies (mgSs), which are assemblages of bacterial strains of the same species, within a microbiome. As demonstrated herein, mgCSTs are associated with demographics such as age and race, as well as vaginal pH and Gram stain assessment of vaginal smears. Importantly, these associations varied between mgCSTs predominated by the same bacterial species. A subset of mgCSTs, including three of the six predominated by G. vaginalis mgSs, as well as a mgSs of L. iners, were associated with a greater likelihood of Amsel bacterial vaginosis diagnosis. This L. iners mgSs, among other functional features, encoded enhanced genetic capabilities for epithelial cell attachment that could facilitate cytotoxin-mediated cell lysis.

The present invention thus demonstrates that MgCSTs are a novel and easily implemented approach to reducing the dimension of complex metagenomic datasets, while maintaining their functional uniqueness. MgCSTs enable investigation of multiple strains of the same species and the functional diversity in that species. Such investigations of functional diversity may be key to unraveling the pathways by which the vaginal microbiome modulates protection to the genital tract. Importantly, the findings presented herein support the hypothesis that functional differences between vaginal microbiomes, including those that may look compositionally similar, are critical considerations in vaginal health.

Based on these findings, the present invention is directed, in non-limiting examples, to methods for characterizing a vaginal microbiome as well as more practical applications such as methods of identifying a subject predisposed to develop bacterial vaginosis, methods of identifying a subject predisposed to re-develop bacterial vaginosis, methods of diagnosing bacterial vaginosis in a subject, methods of treating bacterial vaginosis in a subject, and methods of preventing bacterial vaginosis in a subject.

As summarized above, in a first embodiment the invention is directed to methods of characterizing a vaginal microbiome, comprising obtaining a vaginal microbiome sample from a subject, determining the metagenomic community state type (MgCST) of the sample, and classifying the MgCST as one of MgCSTs 1-27.

In a second embodiment, the invention is directed to methods of identifying a subject predisposed to develop bacterial vaginosis, comprising obtaining a vaginal microbiome sample from a subject, and determining the metagenomic community state type (MgCST) of the sample, wherein when the MgCST of the subject is determined to be a MgCST associated with bacterial vaginosis, the subject is identified as a subject predisposed to develop bacterial vaginosis.

In certain aspects of this embodiment, a MgCST associated with bacterial vaginosis is one or more of MgCSTs 12 and 17-25.

In a third embodiment, the invention is directed to methods of identifying a subject predisposed to re-develop bacterial vaginosis, comprising determining the metagenomic community state type (MgCST) of a vaginal microbiome of a subject after treatment for bacterial vaginosis and classifying it as one of MgCSTs 1-27, wherein when the MgCST of the subject is classified as one or more of MgCSTs 12 and 17-25, the subject is predisposed to re-develop bacterial vaginosis.

In certain aspects of this embodiment, wherein when the MgCST of the subject is classified as one or more of MgCSTs 19 and 22, the subject is predisposed to re-develop bacterial vaginosis.

In a fourth embodiment, the invention is directed to methods of diagnosing bacterial vaginosis in a subject, comprising obtaining a vaginal microbiome sample from a subject, and determining the metagenomic community state type (MgCST) of the sample, wherein when the MgCST of the subject is determined to be a MgCST associated with bacterial vaginosis, the subject is diagnosed as having bacterial vaginosis.

In certain aspects of this embodiment, a MgCST associated with bacterial vaginosis is one or more of MgCSTs 12 and 17-25.

In a fifth embodiment, the invention is directed to methods of treating bacterial vaginosis in a subject, comprising obtaining a vaginal microbiome sample from a subject, determining the metagenomic community state type (MgCST) of the sample, and administering a therapeutically effective amount of an antibacterial agent to the subject when the MgCST of the subject is determined to be a MgCST associated with bacterial vaginosis.

In certain aspects of this embodiment, a MgCST associated with bacterial vaginosis is one or more of MgCSTs 12 and 17-25.

In a sixth embodiment, the invention is directed to methods of preventing bacterial vaginosis in a subject, comprising obtaining a vaginal microbiome sample from a subject, determining the metagenomic community state type (MgCST) of the sample, and administering a prophylactically effective amount of an antibacterial agent to the subject when the MgCST of the subject is determined to be a MgCST associated with bacterial vaginosis.

In certain aspects of this embodiment, a MgCST associated with bacterial vaginosis is one or more of MgCSTs 12 and 17-25.

### Determining Metagenomic Community State Type (MgCST)

The methods of the present invention are each based on determining the metagenomic community state type (MgCST) of the vaginal sample obtained from a subject.

Samples may be obtained from the subject via any suitable means including, but not limited to, self- or clinician-collected vaginal swabs or clinician-collected cervicovaginal lavage.

Once obtained, the sample is placed into preservation buffer (RNALater, C2 Buffer) and frozen at −20° C. or −80° or flash frozen at −80° C. Samples undergo initial processing that includes DNA extraction. Once processed, samples are subject to metagenomic library preparation. The resulting data is then subject to high-through whole genome shotgun sequencing. The final step is assigning or identifying the sample as mgCST 1, 2, 3, 4, 5, 6, 7, 8, 9, 10, 11, 12, 13, 14, 15, 16, 17, 18, 19, 20, 21, 22, 23, 24, 25, 26 or 27.

The required inputs are direct outputs from VIRGO and include the taxonomic abundance table (“summary.Abundance.txt”) and gene abundance table (“summary.NR.abundance.txt”). It is imperative that taxonomic and gene column headings match those output by VIRGO. The expected output is a count table with samples as rows, taxa as columns, and counts normalized by gene length as values. Additional columns indicate the sample mgCST classification and the Yue-Clayton similarity score for all 27 mgCSTs. A heatmap is also produced showing taxon relative abundances in samples, where samples are labeled with assigned mgCSTs Substantial differences may indicate either an incongruence in taxonomic or gene names or the need for an additional mgCST. The classifier is contained in an R script, which is available at https://github.com/ravel-lab/mgCST-classifier. MgSs and mgCST classifications were robust at sampling depths greater than 100,000 reads per sample.

As can be seen, a two-step classifier that assigns metagenomic subspecies and mgCSTs was developed and validated, and it is designed to work in concert with a vaginal non-redundant gene database (VIRGO). This classifier facilitates reproducibility and comparisons across studies.

In each of the relevant embodiments and aspects of the invention as summarized herein, the methods of the invention may further comprise determining one or more of (a) age of the subject, (b) race of the subject, (c) vaginal pH of the subject, and (d) Gram stain assessment of the subject.

In each of the relevant embodiments and aspects of the invention as summarized herein, the methods of the invention may further comprise determining one or more metagenomic subspecies (mgSs) cluster in the sample.

In each of the relevant embodiments and aspects of the invention as summarized herein, the MgCST may be determined by a two-step classifier that assigns one or both of MgSs and mgCSTs. The classifier may be based on mgS composition or abundance, or both. The classifier may use a vaginal non-redundant gene database (VIRGO).

### Subjects

In each of the relevant embodiments and aspects of the invention, the subject is a human, a non-human primate, horse, cow, goat, sheep, a companion animal, such as a dog, cat or rodent, or other mammal.

### Treatment, Prophylaxis and Prevention

As described herein, the present invention is directed to methods of treating bacterial vaginosis in a subject and methods of preventing bacterial vaginosis in a subject, among other equally important goals.

In these methods of treatment and prevention, any antibacterial agent that is therapeutically effective (in methods of treatment) and/or prophylactically effective (in methods of prevention) may be used in the method. Suitable antibacterial agents include, but are not limited to, metronidazole or clindamycin.

The amounts and means for administration of the antibacterial agents will depend on such factors as the MgCST and severity of the infection, demographic information related to the subject, and the selected antibacterial, among other factors. Therefore, dosages and means for administration will be determined by an attending physician or other medical professional.

### III. EXAMPLES

### Methods

Study cohorts. Raw metagenomic data from 1,890 vaginal samples were used in this study. This included publicly available metagenomes including those used in the construction of the vaginal non-redundant gene database, VIRGO (virgo.igs.umaryland.edu, n=342) [29], the University of Maryland Baltimore Human Microbiome Project (UMB-HMP, n=677, PRJNA208535, PRJNA575586, PRJNA797778), the National Institutes of Health Human Microbiome Project (NIH HMP, n=174, phs000228), metagenomes from Li et al. [62] (n=44, PRJEB24147), the Longitudinal Study of Vaginal Flora and Incident STI (LSVF, n=653, dbGaP project phs002367). All samples in LSVF (n=653) and some in UMB-HMP (n=20) had clinical diagnosis information about Amsel-BV. Amsel-BV was diagnosed based on the presence of 3 out of 4 Amsel's criteria [21] and symptomatic Amsel-BV was diagnosed when a woman reported symptoms upon questioning [58]. At the time of these studies, gender identity information was not collected. All women responded to recruiting materials which included “women” or “woman”. In addition, individuals are referred to as women in previous publications, thus individuals are referred to herein as “woman” or “women” to maintain consistency.

Sequence Processing and Bioinformatics. Host reads were removed from all metagenomic sequencing data using BMTagger and the GRCh38 reference genome, and reads were quality filtered using trimmomatic (v0.38, sliding window size 4 bp, Q15, minimum read length: 75 bp) [63]. Metagenomic sequence reads were mapped to VIRGO using bowtie (v1; parameters: —p 16—1 25—fullref—chunkmbs 512—best—strata—m 20—suppress 2,4,5,6,7,8), producing a taxonomic and gene annotation for each read. Samples with fewer than 100,000 mapped reads were removed from the analysis (n=59). The number of reads mapped to a gene was multiplied by the read length (150 bp) and divided by the gene length to produce a coverage value for each gene. Conserved domain and motif searches were performed with CD-SEARCH and the Conserved Domain Database (CDD), using an e-value threshold of 10⁻⁴. The taxonomic composition table generated using VIRGO was run through the vaginal CST classifier VALENCIA [11].

Metagenomic Subspecies. For each species, a presence/absence matrix was constructed from a metagenome which included all genes with at least 0.5× average coverage after normalizing for gene length. Metagenomic subspecies were generated for species present (>75% estimated median number of genes encoded in reference genomes from the Genome Taxonomy Database [64]; data not shown) in >20 samples using binary gene counts and hierarchical clustering with Ward linkage of sample Jaccard distances calculated using the vegdist function from the vegan package (v2.5-5) in R (v. 3.5.2). MgSs were defined using the dynamic hybrid tree cut method (v.1.62-1) and minClusterSize=2 [66]. Heatmaps of gene presence/absence were constructed for each species using the gplots package heatmap.2 function [67] (data not shown). MgSs were tested for associations with low species coverage using logistic regression in which the mgSs was the binary outcome and the log10-transformed coverage of the species was the predictor. Tests were done at the participant level; if a participant had more than one sample and both samples were the same mgSs, only one sample was used, but if the mgSs differed, the samples were included in each. P-values were adjusted for multiple comparisons using Bonferroni correction. Significant dependance was observed in multiple mgSs of Atopobium vaginae, Gardnerella vaginalis, and Lactobacillus iners (data not shown). For these species, the classifiers were built using samples with ≥5.5e5 reads. The cluster stability of each mgSs was evaluated using the clusterboot function of the R package fpc (v 2.2-10) [68, 69] and 100 bootstraps.

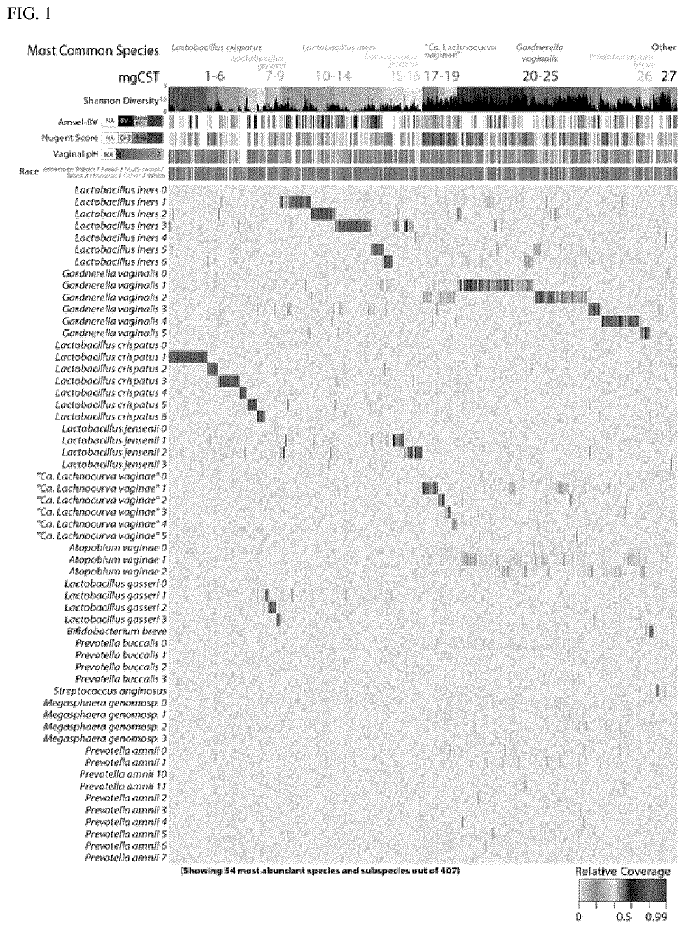

Metagenomic CSTs. Using gene abundance information (normalized by gene length and sequencing depth), the proportion of vaginal species in each sample was estimated. For species that were sub-divided into mgSs, the mgSs proportion in a sample was equal to the proportion of the species in that sample. When a species was present in a sample but with too few genes present to constitute a mgSs (<75% estimated median number of genes encoded in reference genomes), it was labeled as “mgSs 0”. Samples in the resulting compositional table were hierarchically clustered using Jensen-Shannon distances. Clusters were defined using the dynamic hybrid tree cut method (v.1.62-1) [66]. A heatmap for metagenomic CSTs was produced using the gplots package heatmap.2 function (FIG. 1) [67]. For each mgCST, the mgSs most frequently observed (prevalence) and the mgSs with the greatest mean abundance was noted. Cluster stability was evaluated with the clusterboot function from the fpc package (v. 2.2-10) using ward linkage of Jensen-Shannon distances and 100 bootstraps [68]. Cluster stability ≥0.75 is considered high stability.

Statistical analysis of the association between mgCST and age, race, Nugent score, vaginal pH, and BV. For those samples with race, age category, Nugent score category, vaginal pH category, or Amsel-BV diagnoses information (Table 1), the Cochran-Mantel-Haenszel Chi-Squared Test (CMH test, “mantelhaen.test” from the samplesizeCMH R package, v 0.0.0, github.com/pegeler/samplesizeCMH) was used to determine associations with mgCSTs while accounting for source study (the confounding variable). The CMH test evaluates associations between two binary variables (i.e., “mgCST X or not” and “high Nugent score or not”). Tests were done at the participant level; if a participant had more than one sample and both samples were the same mgCST, only one sample was used, but if the mgCSTs differed, the samples were included in each.

Statistical analysis of the association between mgSs, gene clusters, and BV. For both L. iners, associations between mgSs and Amsel-BV were evaluated at the participant level using chi-square analyses which compared the proportion of Amsel-BV positive to negative participants within an mgSs to those in all participants containing any L. iners. For both L. iners and G. vaginalis, associations between gene clusters and Amsel-BV were evaluated at the participant level using chi-square analyses which compared the proportion of Amsel-BV positive to negative participants within a gene cluster to those in all participants containing any L. iners or G. vaginalis gene cluster, respectively. Gene cluster presence in a sample was defined as the presence of ≥30% of genes in a gene cluster.

Longitudinal Stability and Shannon Diversity of L. crispatus mgSs. For participants in the HMP cohort that contributed multiple samples with at least one sample assigned to an L. crispatus mgSs, the Yue-Clayton θ was measured to define microbiota stability for each participant [70]. Here, 16S rRNA gene amplicon sequencing-based CSTs from all samples from a participant [60] were used to produce a reference centroid, and then each sample was compared to that reference (Yue-Clayton's θ). The mean θ for each participant represented the overall microbiota compositional stability. Values closer to 1 indicate high compositional stability. The number of strains in each sample was compared between mgSs using the Wilcoxon signed rank test. Shannon's H diversity index was calculated for each sample using the vegan package diversity function. Shannon Diversity was compared between mgSs using the Wilcoxon signed rank test.

Estimating the number of L. crispatus strains. The number of L. crispatus strains in a mgSs was estimated using a pangenome accumulation curve which was generated by mapping the gene contents of publicly available isolate genome sequences (data not shown) to VIRGO (blastn, threshold: 90% identity, 70% coverage). Bootstrap (n=100) combinations of N (N=1 to 61) isolates were selected and the number of unique L. crispatus Vaginal Orthologous Groups [VOGs; provided in the VIRGO output [29]] encoded in their genomes was determined. An exponential curve relating the number of isolates to the number of VOGs detected was then fit to the resulting data and produced the equation: Y=2057N^{0.14} where Y is the number of L. crispatus VOGs detected, and N is the estimated number of strains. This equation was then used to estimate the number of L. crispatus strains detected in each metagenome based on the observed number of L. crispatus VOGs in each metagenome. The number of strains in each sample was compared between mgSs using the Wilcoxon signed rank test.

Construction of the random forests for mgSs classification. Random forests were constructed for classification of mgSs using the R package randomForestSRC v2.12.1R [71]. For mgSs, a random forest was built for each species (n=28) where the training data contained presence/absence values of genes. Gene presence was defined as above for mgSs. Random forest classification analysis was implemented with all predictors included in a single model. For each mgSs random forest, predictors were all genes in a species. Ten-fold cross-validation (90% of data as training, 10% as testing) was performed wherein each training set was used to build and tune a random forest model using tune “tune.rfsrc”. A random forest model using optimal parameters was then used to predict mgSs classifications for the test set and out-of-bag error estimates (misclassification error) are reported. The overall misclassification error is the average misclassification error from each fold and the “correct” assignment is based on original hierarchical clustering assignment. The final models included all data and the optimal tuning parameters determined for that species. For mgSs assignment, the mgSs which provides the highest probability (based on the proportion of votes in the tree) is used for assignment. The user is provided both the assignment, as well as the probability of that assignment as a measure of confidence.

Construction of the a nearest centroid classifier for mgCSTs. Using mgCSTs as defined above, reference centroids were produced using the mean relative abundances of each mgSs in a mgCST. For classification, the similarity of a sample to the reference centroids is determined using Yue-Clayton's θ [70]. Compared to Jensen-Shannon, the Yue-Clayton θ measure depends more on the high relative abundance metagenomic subspecies than those at lower relative abundances. Samples are assigned to the mgCST to which they bear the highest similarity and the degree of similarity to that mgCST can be taken as a measure of confidence in the assignment. Ten-fold cross validation was applied wherein each training set was used to build “reference” centroids and each test set was used for assignment. The misclassification error was determined by subtracting the number of correct assignments (based on original hierarchical clustering assignment) divided by the total number of assignments from 1. The overall misclassification error is the average of misclassification error from each fold.

Running the mgCST classifier. The required inputs are direct outputs from VIRGO [29] and include the taxonomic abundance table (“summary.Abundance.txt”) and gene abundance table (“summary.NR.abundance.txt”). It is imperative that taxonomic and gene column headings match those output by VIRGO. The expected output is a count table with samples as rows, taxa as columns, and counts normalized by gene length as values. Additional columns indicate the sample mgCST classification and the Yue-Clayton similarity score for all 27 mgCSTs. A heatmap is also produced showing taxon relative abundances in samples, where samples are labeled with assigned mgCSTs. Substantial differences may indicate either an incongruence in taxonomic or gene names or the need for an additional mgCST. The classifier is contained in an R script, which is available at github.com/ravel-lab/mgCST-classifier. The classifier is a random forest model+Yue-Clayton theta models. Thus, the classifier requires the training sets available here: https://figshare.com/account/home#/projects.

Validation of the mgCST classifier in external datasets. Three external, publicly available vaginal metagenome datasets were used to validate the generalizability of mgCST assignments beyond the training dataset. ENA PRJEB34536 [72]. NIH PRJNA576566 [73], and NIH PRJNA779415 [74]. Briefly, host reads were removed using BMTagger [75] and the GRch38_p12 human reference genome. Ribosomal RNA reads were removed using sortmerna [76] and reads were quality filtered using fastp [77] with a minimum length of 50 bp, and a mean quality of 20 in a sliding window of 4 bp. Remaining reads were processed through VIRGO using default settings (virgo.igs.umaryland.edu) [29] and summary tables were used to assign mgSs and mgCSTs with the mgCST Classifier. Generalizability of the mgSs and mgCST reference datasets is illustrated using probability of assignment by the mgSs random forest classifier and the Yue-Clayton similarity scores of assigned mgCSTs. All bioinformatic and statistical analyses are available in R Markdown notebooks (File_S7 and File S8).

### Results

Metagenomic Community State Types (mgCST) of the Vaginal Microbiome.

The within-species bacterial genomic diversity was evaluated in 1,890 vaginal metagenomes of reproductive-age participants from 1,024 mostly North American women (98.7% of samples) (Table 1). Vaginal metagenomes derived from five cohort studies as well as metagenomes generated to build the vaginal non-redundant gene database (VIRGO, [29]) were used to construct mgCSTs as described above. In total, 135 metagenomic subspecies (mgSs) from 28 species were identified by hierarchical clustering of species-specific gene presence/absence profiles (data not shown). Subsequent hierarchical clustering of samples based on mgSs compositional data produced 27 mgCSTs (Table 2). Cluster stability was ≥0.75 for most mgCSTs (Table 2). MgCSTs consisted of mgSs from commonly observed vaginal species including L. crispatus (mgCST 1-6, 19% of samples), L. gasseri (mgCST 7-9, 3% of samples), L. iners (mgCST 10-14, 23% of samples), L. jensenii (mgCST 15 and 16, 4.6% of samples), “Ca. Lachnocurva vaginae” (mgCST 17-19, 7.5% of samples), G. vaginalis (mgCST 20-25, 36.3% of samples) and Bifidobacterium breve (mgCST 26, 0.74% of samples) (FIG. 1). MgCST 27 (5.5% of samples) contained less-common species such as Streptococcus anginosus or had no predominant taxon. MgCST 2 (n=39 samples from 26 women), mgCST 14 (n=34 samples from 25 women), and mgCST 21 (n=37 samples from 21 women), were only comprised of samples from reproductive aged women in Alabama enrolled in the UMB-HMP cohort (Table 2). Metagenomic CSTs expand amplicon-based CSTs as multiple mgCSTs are predominated by the same species, but a different mgSs of that species (Table 2).

TABLE 1
Demographic information for all women included in this study. For
each data category (Age, Race, etc.), the total number of women
and samples are notes. Percentages represent the proportions of
women or samples within each category. Some women contributed
multiple samples.

| | Number | | Number | |
|---|---|---|---|---|
| | of Women | | of Samples | |
| | With | | With | |
| | Category- | | Category | |
| | Specific | | Specific | |
| Categories | Data | Percentage | Data | Percentage |
| Metagenomic | 1,017 | 100.0 | 1,890 | 100.0 |
| Data Source | | | | |
| UMB-HMP | 124 | 12.2 | 515 | 27.2 |
| Li et al. | 44 | 4.3 | 44 | 2.3 |
| LSVF | 585 | 57.5 | 653 | 34.6 |
| NIH-HMP | 76 | 7.5 | 174 | 9.2 |
| VMRC | 40 | 3.9 | 162 | 8.6 |
| VIRGO | 148 | 14.6 | 342 | 18.1 |
| Age (yo) | 897 | 100.0 | 1,623 | 100.0 |
| 15-20 | 283 | 31.5 | 410 | 25.3 |
| 21-25 | 229 | 25.5 | 436 | 26.9 |
| 26-30 | 188 | 21.0 | 362 | 22.3 |
| 31-35 | 102 | 11.4 | 223 | 13.7 |
| 36-40 | 65 | 7.2 | 125 | 7.7 |
| 41-45 | 30 | 3.3 | 67 | 4.1 |
| Race | 858 | 100.0 | 1,441 | 100.0 |
| Asian | 54 | 6.3 | 66 | 4.6 |
| Black or African | 610 | 71.1 | 968 | 67.2 |
| America | | | | |
| Hispanic or Latino | 19 | 2.2 | 47 | 3.3 |
| Other | 6 | 0.7 | 9 | 0.6 |
| White or Caucasian | 169 | 19.7 | 351 | 24.4 |
| Nugent Category | 968 | 100.0 | 1,623 | 100.0 |
| 0-3 | 469 | 48.5 | 931 | 57.4 |
| 4-6 | 194 | 20.0 | 255 | 15.7 |
| 7-10 | 305 | 31.5 | 437 | 26.9 |
| Vaginal pH Category | 874 | 100.0 | 1,362 | 100.0 |
| Low (pH <4.5) | 273 | 31.2 | 491 | 36.0 |
| High (pH ≥4.5) | 601 | 68.8 | 871 | 64.0 |
| Amsel-BV Diagnosis | 627 | 100.0 | 673 | 100.0 |
| Positive | 289 | 46.1 | 308 | 45.8 |
| Negative | 338 | 53.9 | 365 | 54.2 |
| Symptomatic | 289 | 100.0 | 308 | 100.0 |
| Amsel-BV | | | | |
| Asymptomatic | 253 | 87.5 | 271 | 88.0 |
| Symptomatic | 36 | 12.5 | 37 | 12.0 |

Vaginal Non-redundant [symbol] Database (VIRGO, virgo.lgs. [symbol] .edu) [29], the University of Maryland and Baltimore Human Microbiome Project (UMB-HMP), PRJNA208535, PRJNA575586, PRJNA797778), the National Institutes of Health, Human Microbiome Project (NIH-HMP, pbs000228), Li et al. [60] (PRJEB24147], the Longitudinal Study of Vaginal Flora and Incident STI (LSVP, dbGaP project pbs002367). [symbol]
indicates data missing or illegible when filed

TABLE 2
Twenty-seven metagenomic community state types (mgCSTs) were defined for the vaginal microbiome. Cluster stability
of mgCSTs was evaluated using bootstrap analysis. Most mgCSTs comprise of samples from different source studies.

| | | | Number of | Number of | | | [symbol] of Samples from [symbol] | | | | | |
|---|---|---|---|---|---|---|---|---|---|---|---|---|
| MgCST | [symbol] | [symbol] | [symbol] | [symbol] | [symbol] | [symbol] | [symbol] | [symbol] | [symbol] | [symbol] | [symbol] | [symbol] |
| 1 | Lactobacillus [symbol] 1 | Lactobacillus [symbol] 1 | [symbol] | [symbol] | [symbol] | [symbol] | [symbol] | [symbol] | [symbol] | [symbol] | [symbol] | [symbol] |
| 2 | Lactobacillus [symbol] 2 | Lactobacillus [symbol] 2 | [symbol] | [symbol] | [symbol] | [symbol] | [symbol] | [symbol] | [symbol] | [symbol] | [symbol] | [symbol] |
| 3 | Lactobacillus [symbol] 3 | Lactobacillus [symbol] 3 | [symbol] | [symbol] | [symbol] | [symbol] | [symbol] | [symbol] | [symbol] | [symbol] | [symbol] | [symbol] |
| 4 | Lactobacillus [symbol] 4 | Lactobacillus [symbol] 4 | [symbol] | [symbol] | [symbol] | [symbol] | [symbol] | [symbol] | [symbol] | [symbol] | [symbol] | [symbol] |
| 5 | Lactobacillus [symbol] 5 | Lactobacillus [symbol] 5 | [symbol] | [symbol] | [symbol] | [symbol] | [symbol] | [symbol] | [symbol] | [symbol] | [symbol] | [symbol] |
| 6 | Lactobacillus [symbol] 6 | Lactobacillus [symbol] 6 | [symbol] | [symbol] | [symbol] | [symbol] | [symbol] | [symbol] | [symbol] | [symbol] | [symbol] | [symbol] |
| 7 | Lactobacillus [symbol] 1 | Lactobacillus [symbol] 1 | [symbol] | [symbol] | [symbol] | [symbol] | [symbol] | [symbol] | [symbol] | [symbol] | [symbol] | [symbol] |
| 8 | Lactobacillus [symbol] 2 | Lactobacillus [symbol] 2 | [symbol] | [symbol] | [symbol] | [symbol] | [symbol] | [symbol] | [symbol] | [symbol] | [symbol] | [symbol] |
| 9 | Lactobacillus [symbol] 3 | Lactobacillus [symbol] 3 | [symbol] | [symbol] | [symbol] | [symbol] | [symbol] | [symbol] | [symbol] | [symbol] | [symbol] | [symbol] |
| 10 | Lactobacillus [symbol] 1 | Lactobacillus [symbol] 1 | [symbol] | [symbol] | [symbol] | [symbol] | [symbol] | [symbol] | [symbol] | [symbol] | [symbol] | [symbol] |
| 11 | Lactobacillus [symbol] 2 | Lactobacillus [symbol] 2 | [symbol] | [symbol] | [symbol] | [symbol] | [symbol] | [symbol] | [symbol] | [symbol] | [symbol] | [symbol] |
| 12 | Lactobacillus [symbol] 3 | Lactobacillus [symbol] 3 | [symbol] | [symbol] | [symbol] | [symbol] | [symbol] | [symbol] | [symbol] | [symbol] | [symbol] | [symbol] |
| 13 | Lactobacillus [symbol] 5 | Lactobacillus [symbol] 5 | [symbol] | [symbol] | [symbol] | [symbol] | [symbol] | [symbol] | [symbol] | [symbol] | [symbol] | [symbol] |
| 14 | Lactobacillus [symbol] 6 | Lactobacillus [symbol] 6 | [symbol] | [symbol] | [symbol] | [symbol] | [symbol] | [symbol] | [symbol] | [symbol] | [symbol] | [symbol] |
| 15 | Lactobacillus [symbol] | Lactobacillus [symbol] | [symbol] | [symbol] | [symbol] | [symbol] | [symbol] | [symbol] | [symbol] | [symbol] | [symbol] | [symbol] |
| 16 | Lactobacillus [symbol] | Lactobacillus [symbol] | [symbol] | [symbol] | [symbol] | [symbol] | [symbol] | [symbol] | [symbol] | [symbol] | [symbol] | [symbol] |
| 17 | “Ca.” Lachnocurva vaginae 1 | “Ca.” Lachnocurva vaginae 1 | [symbol] | [symbol] | [symbol] | [symbol] | [symbol] | [symbol] | [symbol] | [symbol] | [symbol] | [symbol] |
| 18 | “Ca.” Lachnocurva vaginae 2 | “Ca.” Lachnocurva vaginae 2 | [symbol] | [symbol] | [symbol] | [symbol] | [symbol] | [symbol] | [symbol] | [symbol] | [symbol] | [symbol] |
| 19 | “Ca.” Lachnocurva vaginae 3 | “Ca.” Lachnocurva vaginae 3 | [symbol] | [symbol] | [symbol] | [symbol] | [symbol] | [symbol] | [symbol] | [symbol] | [symbol] | [symbol] |
| 20 | Gardnerella vaginalis [symbol] | Gardnerella vaginalis [symbol] | [symbol] | [symbol] | [symbol] | [symbol] | [symbol] | [symbol] | [symbol] | [symbol] | [symbol] | [symbol] |
| 21 | Gardnerella vaginalis [symbol] | Gardnerella vaginalis [symbol] | [symbol] | [symbol] | [symbol] | [symbol] | [symbol] | [symbol] | [symbol] | [symbol] | [symbol] | [symbol] |
| 22 | Gardnerella vaginalis [symbol] | [symbol] 4 | [symbol] | [symbol] | [symbol] | [symbol] | [symbol] | [symbol] | [symbol] | [symbol] | [symbol] | [symbol] |
| 23 | Gardnerella vaginalis [symbol] | Gardnerella vaginalis [symbol] | [symbol] | [symbol] | [symbol] | [symbol] | [symbol] | [symbol] | [symbol] | [symbol] | [symbol] | [symbol] |
| 24 | Gardnerella vaginalis [symbol] | Gardnerella vaginalis [symbol] | [symbol] | [symbol] | [symbol] | [symbol] | [symbol] | [symbol] | [symbol] | [symbol] | [symbol] | [symbol] |
| 25 | Gardnerella vaginalis [symbol] | Gardnerella vaginalis [symbol] | [symbol] | [symbol] | [symbol] | [symbol] | [symbol] | [symbol] | [symbol] | [symbol] | [symbol] | [symbol] |
| 26 | [symbol] | [symbol] | [symbol] | [symbol] | [symbol] | [symbol] | [symbol] | [symbol] | [symbol] | [symbol] | [symbol] | [symbol] |
| 27 | [symbol] | [symbol] | [symbol] | [symbol] | [symbol] | [symbol] | [symbol] | [symbol] | [symbol] | [symbol] | [symbol] | [symbol] |

[symbol]

[symbol]

indicates data missing or illegible when filed

Vaginal mgCSTs and Demographics.

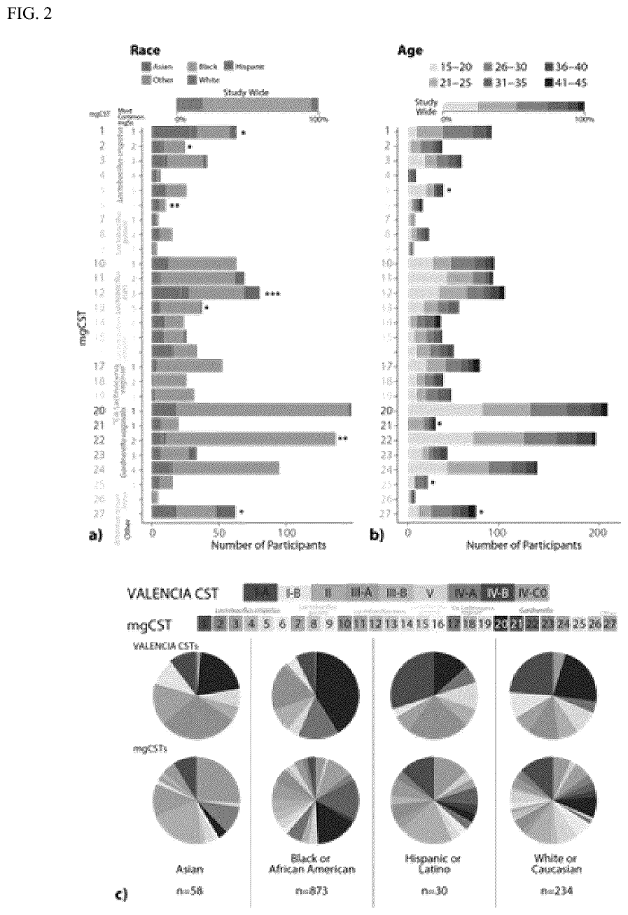

Race and Age. Race information was available for 1,441 samples reported by 858 women. Most women identified as either Black (71%) or White (20%), and the remainder as Asian (6.3%), Hispanic (2.2%), or other (<1%) (Table 1). Age was also reported for 1,623 samples from 897 individuals and ranged from 15-45 years old. After adjusting for between-cohort heterogeneity, certain races and age categories were associated with mgCSTs (FIG. 2). The vaginal microbiomes of Black women were more likely to be classified as G. vaginalis mgCST 22 (p=0.0006) and least likely to be in L. crispatus mgCST 1 (p=0.005) as compared with microbiomes for other races (data not shown). Microbiomes classified as mgCST 6 were more likely to be from White women than other races (p=0.002). L. iners mgCST 12 was most common among Hispanic and Asian women (p=0.0001), and L. iners mgCSTs 10 and 14 were absent in Asian women (FIG. 2c). MgCSTs predominated by “Ca. Lachnocurva vaginae” (mgCSTs 17-19) were also not observed in Asian women, consistent with previous reports on that species [11]. In mgCST 27, women were less likely to be Black (p=0.01) and more likely to be in the oldest age category (41-45, p=0.04) as compared with other mgCSTs.

Nugent Scores and Vaginal pH. Of the 968 women for which Nugent scores were available, 48% had low Nugent scores (0-3), 20% had intermediate scores (4-6), and 32% had high scores (7-10) (Table 1). The Nugent scoring system is a Gram stain scoring method for vaginal smears that helps diagnose bacterial vaginosis. A vaginal smear is plated on a microscopic slide and examined for the presence of three types of bacteria: Lactobacillus, Gardnerella, and curved gram rods. Each bacteria type is scored based on the number of bacteria counted, and the three scores are added together for a total score between 0 and 10. A score of 0-3 is considered negative for BV, 4-6 is intermediate, and 7-10 is positive for BV.

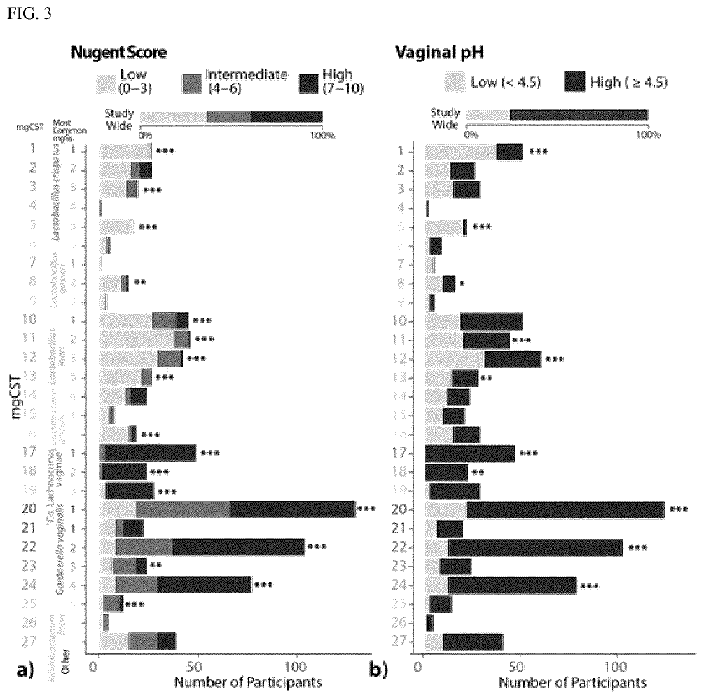

Vaginal pH was also available for 979 women and of these 31% had low pH <4.5, and 69% had high pH ≥4.5 (Table 1). Both Nugent score and vaginal pH were associated with mgCSTs after adjusting for between-cohort heterogeneity (FIG. 3). Of all L. crispatus mgCSTs, mgCST 2 had the most representation of different Nugent categories, with 61%, 14%, and 25% of samples having low, intermediate, or high Nugent scores, respectively (FIG. 3a). Communities predominant in “Ca. Lachnocurva vaginae” mgCSTs 17, 18, and 19 had the highest percentages of high Nugent scores (7-10), (94%, 96%, and 87% of samples, respectively); and these mgCSTs were also associated with high vaginal pH (p=6.3 e⁻⁷, FIG. 3b). Notably, intermediate Nugent scores were common among G. vaginalis predominated mgCSTs, especially in mgCSTs 25 (69% of samples).

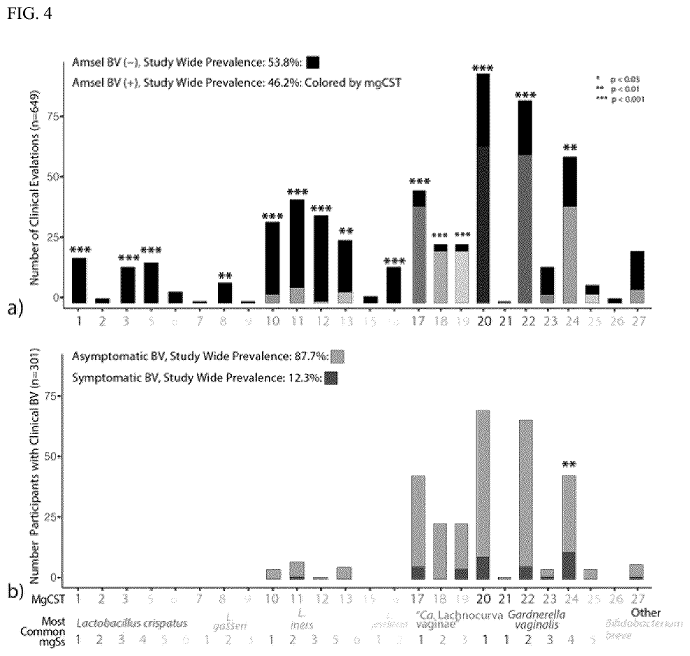

Amsel-BV and Vaginal Symptoms. Of 627 women, each with a vaginal sample and same-day clinical examination data (n=607 from LSVF cohort, n=20 from HMP cohort), 40.3% had asymptomatic Amsel-BV and 5.5% had symptomatic Amsel-BV diagnoses. Twelve percent of Amsel-BV cases were symptomatic. Diagnosis of Amsel-BV was associated with mgCSTs (FIG. 4a). There were no Amsel-BV diagnoses in mgCSTs predominated by L. crispatus, L. jensenii, or L. gasseri. L. iners predominated mgCSTs 10-13 were negatively associated Amsel-BV diagnoses (p=9.6e⁻⁴) but contained some positive Amsel-BV diagnoses in mgCSTs 10, 11, and 13 (11%, 15%, 18% of women, respectively) (FIG. 4a). L. iners mgCST 12 contained only a single (asymptomatic) positive Amsel-BV diagnosis out of 39 women. Women with “Ca. Lachnocurva vaginae” mgCSTs 17-19 were more likely to have been diagnosed with Amsel-BV (87%, 88%, and 89%, respectively, p=1.8e⁻⁵). G. vaginalis predominated mgCSTs 20, 22, and 24 also had significantly more positive Amsel-BV diagnoses than the study-wide proportion (69%, 73%, and 66%, respectively, p=1.5e⁻³), while 75% of G. vaginalis predominated mgCST 23 samples were Amsel-BV negative (p=0.09). MgCST 24 contained significantly more symptomatic cases than expected (26% of 43 individuals, p=0.008, FIG. 4b). Though not statistically significant, “('a. Lachnocurva vaginae” mgCST 19 also may have a higher-than-expected proportion of symptomatic Amsel-BV cases (17.4%).

Functional Potential of mgCSTs and Metagenomic Subspecies.

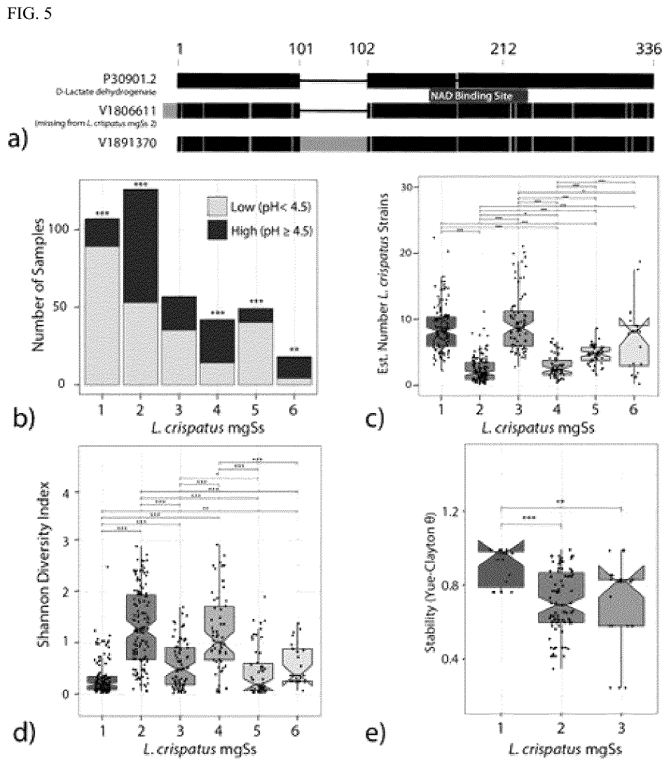

L. crispatus mgCSTs differ by species diversity, stability, and the potential to produce D-lactic acid. L. crispatus is known to produce both L- and D-lactic acid, which acidifies the vaginal environment and confers protective properties [4, 10, 31, 32]. Metagenomic analyses revealed differences among L. crispatus mgSs. First, VIRGO identified two L- and two D-lactate dehydrogenase genes in L. crispatus. All genes were present in L. crispatus mgSs except for mgSs 2. MgSs 2 was missing a D-lactate dehydrogenase gene (V1806611) that has 96.1% identity to a functionally validated ortholog, P30901.2 (FIG. 5a) [33]. The other D-lactate dehydrogenase, V1891370, is found in all L. crispatus mgSs but only 82.4% identical to P30901.2 because it contains a 55 aa insertion after V101 (position in P30901.2) and a point mutation at position 218 (D218Y) located within a NAD binding site domain, the functional consequences of which are unknown. Because V1806611 is most similar to P30901.2, its absence may influence the production of D-lactic acid. Second, in mgSs 2, 4, and 6 60-70% of samples were significantly more likely to be from the high vaginal pH category compared to 41% of samples with any L. crispatus mgSs (FIG. 5b). Third, mgSs 2 had significantly fewer estimated numbers of L. crispatus strains compared to other L. crispatus mgSs (FIG. 5c). Fourth, mgSs 2 and 4 were on average more compositionally diverse than other mgSs (FIG. 5d). Lastly, the mgSs I was significantly more longitudinally stable than mgCST 2 or 3 as defined using Yue-Clayton's θ (FIG. 5e). Overall, these differences reveal important genetic and functional differences among vaginal microbiomes primarily comprised of L. crispatus which could be important in understanding the role of L. crispatus in the vaginal microbiome.

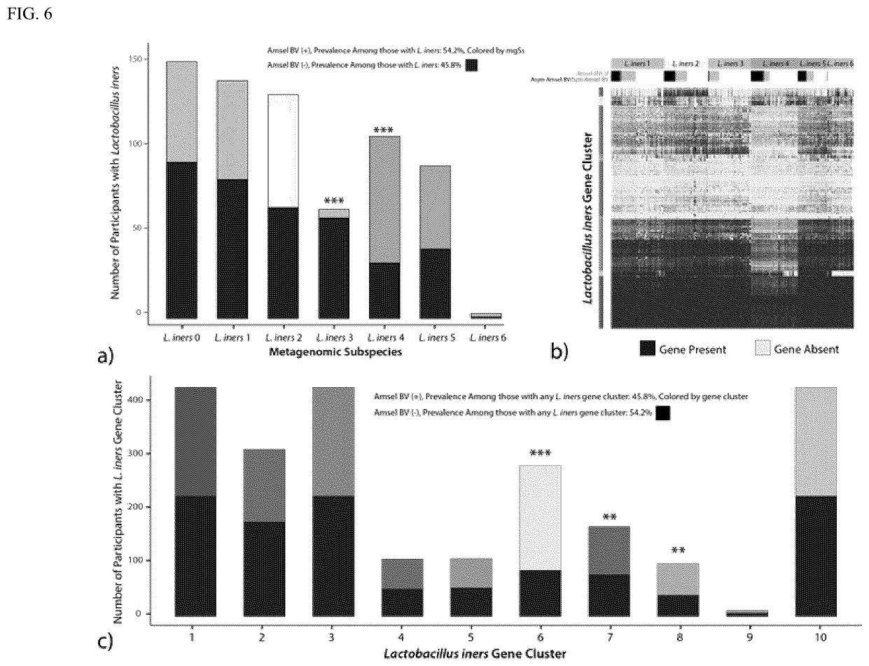

L. iners metagenomic subspecies are associated with Amsel-BV diagnoses. The role of L. iners in the vaginal microbiome is not fully understood because it has been implicated in both healthy and BV states [34]. Sixty-five percent of samples containing L. iners mgSs 4 were positive Amsel-BV cases which is significantly greater than the proportion of cases harboring any L. iners mgSs (45.8%, p=1.1e⁻⁶, FIG. 6a). Conversely, L. iners mgSs 3, which predominates mgCST 12, was associated with negative Amsel-BV diagnoses (86% Amsel-BV negative, p<0.0001). L. iners is represented by six mgSs of which 5 predominated an mgCST; L. iners mgSs 4 did not (FIG. 1). Instead, L. iners mgSs 4 was present in relatively lower abundances (median: 1.2%, IQR: 1.9%) in 257 microbiomes from BV-like mgCSTs 16, 17, and 18, 19 and 24.

Next, whether L. iners genes were associated with Amsel-BV was evaluated. Most samples in L. iners mgSs 4 contained genes from cluster 6 (yellow gene cluster, FIG. 6b). There were significantly more positive Amsel-BV diagnoses among participants containing L. iners gene cluster 6 (69.4%, p=2.1e⁻¹⁵), 7 (53.9%, p=0.004), or 8 (60.2%, p=0.036) compared to samples containing any other L. iners gene cluster (45.8%, FIG. 6c). Gene products unique to L. iners gene cluster 6 had significant similarity to virulence factors that could contribute to L. iners ability to thrive in dynamic vaginal states. Such factors include serine/threonine-protein kinases (STPKs), SHIRT domains known as “periscope proteins” which regulate bacterial cell surface interactions related to host colonization [35], CRISPR-cas, β-lactamase and multidrug resistance (MATE), and bacterocin exporters (data not shown). Gene products in cluster 7 included ParM, which plays a vital role in plasmid segregation, pre-protein translocation and membrane anchoring (SecA, SecY, sortase), defense mechanism beta-lytic metallopeptidase, and mucin-binding and internalin proteins. In Listeria monocytogenes, internalin A mediates adhesion to epithelial cells and host cell invasion [36]. Phage-like proteins in gene group 8 suggest the presence of mobile elements. The presence of the highly-conserved L. iners pore-forming cytolysin, inerolysin [37], did not differ by mgSs.

### Diversity of Gardnerella Vaginalis Genomospecies is Associated With an Increase in Virulence Factors.

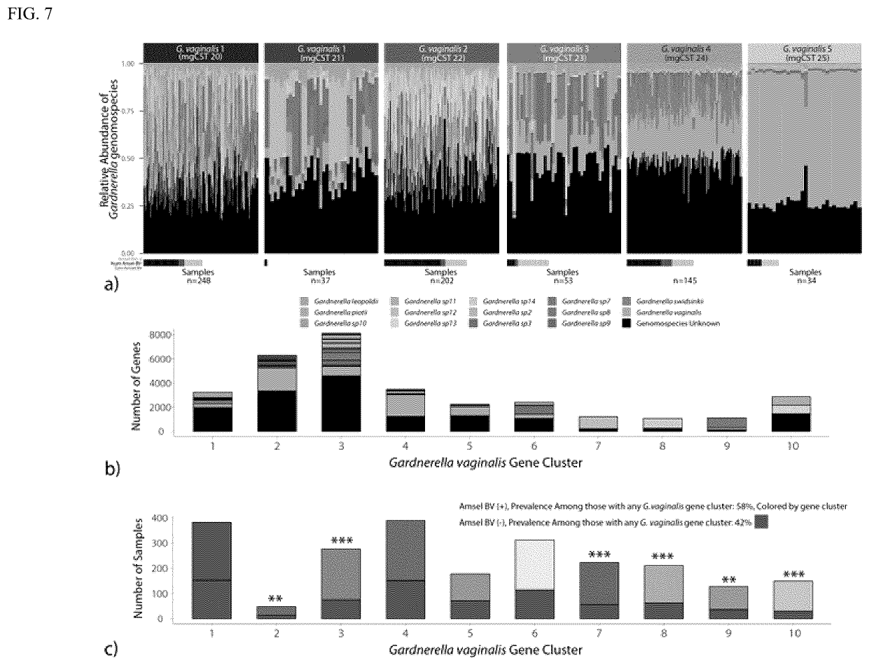

As previously mentioned, positive Amsel-BV diagnoses were common in G. vaginalis mgCSTs 20, 22, and 24, while mgCST 23 contained more relatively more Amsel-BV negative samples than positive Amsel-BV (FIG. 4). Symptomatic BV cases were more common in mgCST 24 than in all other mgCSTs. By mapping the available genomes of various Gardnerella genomospecies [38] to VIRGO, it was determined that each G. vaginalis mgSs consists of a unique combination of Gardnerella genomospecies (FIG. 7a). Compared to other G. vaginalis mgCSTs, mgCSTs 20-22 contain a greater number of Gardnerella genomospecies than mgCSTs 23-25. MgCST 24 samples are predominated by G. vaginalis mgSs 4 and largely consists of G. swidsinkii and G. vaginalis genes. This suggests the diversity and types of Gardnerella genomospecies may be important determinants of the pathogenicity of mgCSTs. G. vaginalis gene clusters contain different proportions of Gardnerella genomospecies (FIG. 7b). Certain gene clusters are associated with Amsel-BV diagnoses, especially gene cluster 10 (FIG. 7c), which is primarily comprised of genes from Gardnerella sp 11 and sp 13 (FIG. 7b) and encodes a vaginolysin (V1099403), a hemolysin (V1099398), the muralytic enzyme precursor Rpf2 (V1385511), a vancomycin resistance protein (V1099665), and glycogen debranching enzymes pullulanase (V1313000) and oligo-16-glucosidase (V1195401).

Automated Classification of mgCSTs Using Random Forest Models.

Random forest models were built for each of the 135 mgSs identified and used to perform mgSs assignments as described above. There was good concordance between mgSs assigned by hierarchical clustering of Jensen-Shannon distances and random forest based assignments with κ>0.8 for most species (data not shown). Ten-fold cross validation of the classifier revealed the misclassification error for mgSs assignment ranged from 0-30% (data not shown). The error estimates for most major vaginal taxa were near or less than 10%, with L. gasseri having the lowest (2.2%). L. iners consistently provided higher misclassification error estimates (20%) regardless of attempts to fine-tune the model and was likely the result of high genetic homogeneity between and heterogeneity within L. iners mgSs. Following assignment of mgSs, mgCSTs were assigned using the nearest centroid classification method, as previously used for vaginal taxonomy-based community state type assignments [11]. Good concordance was observed between mgCSTs assigned by hierarchical clustering of Jensen-Shannon distances and nearest centroid based assignments (κ=0.78, data not shown). Ten-fold cross validation of centroid classification revealed the mean classification error was 9.6%, with some mgCSTs classified more accurately than others (data not shown).

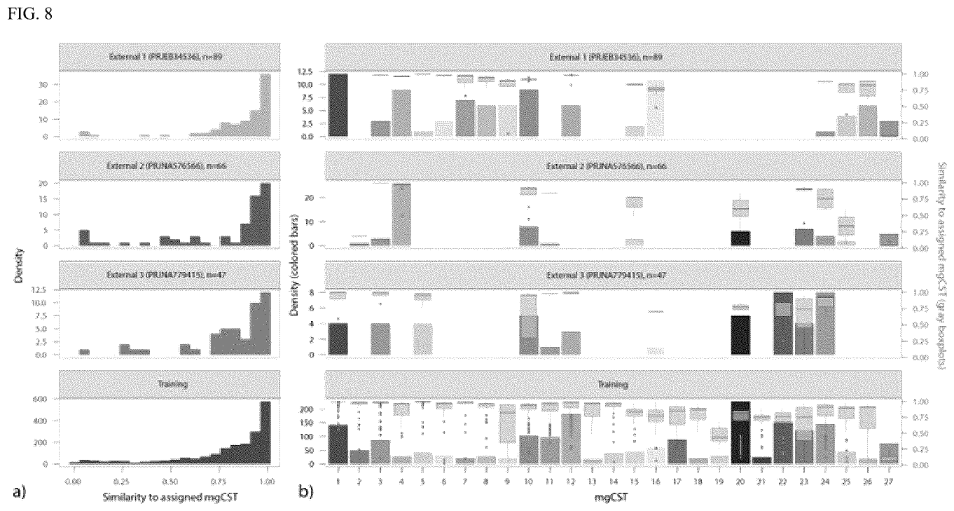

Three external, publicly available metagenomic datasets illustrate generalizability of the mgCSTs assignment (FIG. 8). Most samples produced similarity scores of >0.5 indicating high similarity to the reference centroid of the assigned mgCST (FIG. 8a). Samples in each dataset were distributed across mgCSTs (FIG. 8b, primary y-axis). In all datasets, the lowest similarity scores were observed in mgCST 27 (FIG. 8b, secondary y-axis).

The source code for the mgCST classifier is an R script and is available at https://github.com/ravel-lab/mgCST-classifier and uses direct outputs from VIRGO.

Bacterial Vaginosis (BV) and Recurrence (rBV).

As described above, 27 mgCSTs were characterized from 1,898 samples and each was dominated by different strain combinations of commonly observed vaginal bacteria (FIG. 1). Previously, vaginal microbiota clustered in to community state types using 16S rRNA gene amplicon-based taxonomy [1]. The mgCSTs presented herein utilize both taxonomic and functional information contained in gene groups of each major species in the vaginal microbiome, and associate with the prevalence of Amsel-BV, Nugent scores [25], vaginal pH, and race (FIG. 1) [82]. Thus, mgCSTs distinguish vaginal microbiomes at higher resolution compared to those identified by traditional 16S rRNA gene amplicon profiles and at least nine functionally distinct forms of BV exist.

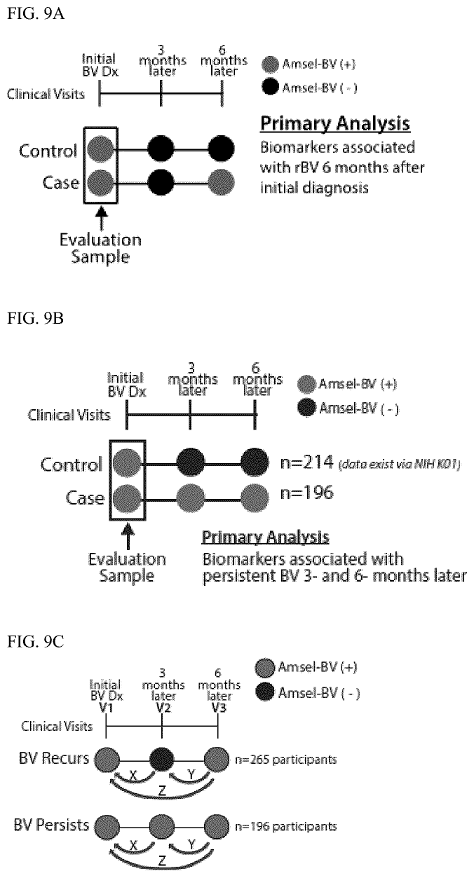

(i) Metronidazole treatment does not prevent recurrence. Certain BV-like mgCSTs are prognostic of recurrent Amsel-BV, namely CLV 19 and Gv 22. A retrospective case-control analysis within the Longitudinal Study of Vaginal Flora [24], a longitudinal observational study recruited reproductive age, nonpregnant women presenting for routine health care visits at 1 of 12 health department clinics in Birmingham, Alabama from August 1999 to February 2002, was performed. Clinical evaluations, participant surveys, and cervicovaginal lavages (CVL) were collected at each visit. Bacterial Vaginosis (BV) was diagnosed by the observation of 3 of 4 Amsel's criteria [21]. In the analysis, the primary outcomes of interest were BV resolution (controls, n=232) or recurrence (cases, n=402) after an index BV event wherein a participant received a clinical diagnosis of Amsel-BV during the study period, irrespective of patient-reported BV signs (foul odor, vaginal discharge, itching, or burning) (FIG. 9A). Episodes of recurrent BV were defined as a negative Amsel-BV evaluation at the visit after the index visit (occurring three months later) and a subsequent positive Amsel-BV diagnosis three months thereafter (six months since the initial visit). BV resolution was defined as three consecutive visits after the index BV event (over six months) with negative Amsel-BV diagnoses. Participants that reported symptoms received metronidazole treatment, the recommended standard-of-care for BV since 1982 [93], and this occurred in 82 and 77% of people that experienced BV resolution or recurrence, respectively.

In this multinomial model accounting for known BV risk factors: race [83], prior history of BV [84,85], and smoking status [86], as well as personal hygiene and sexual practices during the interval prior to the outcome, receiving metronidazole treatment was not associated with BV resolution and, in fact, trended towards lower odds of resolution compared to recurrence (OR: 0.6, 95% CI: 0.3-1.1), emphasizing the long-term ineffectiveness of the current CDC standard-of-care guidelines for BV [58,84,87,90].

Comparatively, certain mgCSTs at the index visit, namely CLV 19 and Gv 22, were associated with 10 and nearly 20-fold increased odds of BV recurrence compared to Lactobacillus-dominated microbiomes, respectively, while Gv mgCSTs 23 and 25 were not related to recurrence. These findings remain significant when accounting for hygiene practices and sexual behaviors following the index visit and imply that reinfection through sexual habits is unlikely to explain these associations. These data highlight for the first time that mgCST-based definitions of BV can identify recurrent BV, and this presents an opportunity to develop diagnostic tests and novel treatments tailored to prevent recurrence. Leading hypotheses about the recurrence of BV include metronidazole resistance through ferredoxin/ferredoxin-NADP reductase (FNR) and nitroimidazole reductase, the production of polymicrobial biofilms, and sexual partner reinfection [88-92]. Thus far, genomic investigations have revealed that Gv 22 differs from other Gv mgCSTs in its potential to bind host epithelia and has added potential for mucin degradation through Gardnerella sp. 11 and 13 that are unique to mgCST 22 (FIG. 7a).

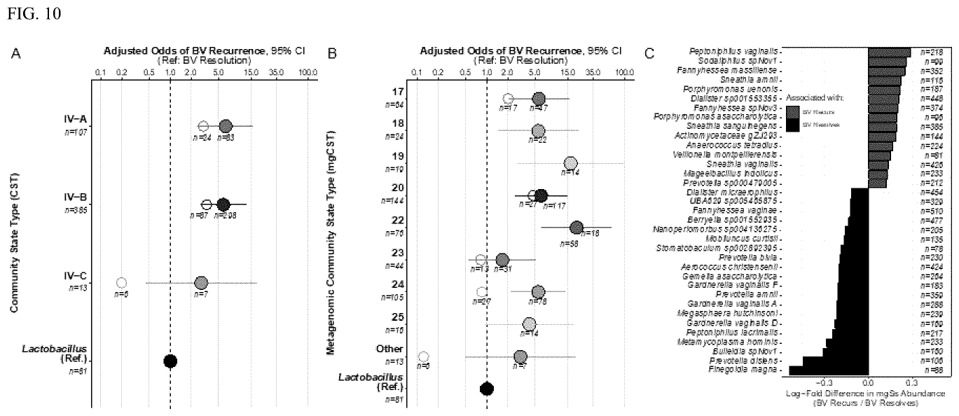

(ii) Functionally distinct vaginal microbiomes predict BV recurrence and resolution. Using traditional amplicon-sequencing based characterizations, the odds of recurrence were 6-fold greater than resolution in all non-Lactobacillus predominated communities compared to Lactobacillus abundant microbiota (FIG. 10A). These results are expected yet, because CSTs are not functionally informed through gene content, are unable to lead to novel insights about types of BV that may be more prone to recurrence via mechanisms such as treatment resistance. Application of the mgCST approach reveals a deeper story: certain types of non-Lactobacillus microbiomes are highly associated with recurrence (FIG. 10B). 90% of participants with mgCSTs 19 (Ca. Lachnocurva vaginae 3) or 22 (G. vaginalis C) experienced recurrence, and together mgCSTs 19 and 22 accounted for 26% of all index BV events (163 of 634) in this study. Furthermore, those with mgCST 23 (G. vaginalis D) appeared to have low risk of recurrence where the odds of recurrence were not statistically different from Lactobacillus-predominated communities (aOR: 1.6, 95% CI: 0.6-4). Of these 44 participants, 70% did not receive treatment, and of those that did (n=13), fewer than half experienced resolution demonstrating evidence that the low risk of recurrence attributed to mgCST 23 was not because of metronidazole treatment. Interestingly, in mgCST 24, the risk of recurrence was attenuated with metronidazole treatment. Results of the mgCST analysis are substantiated at the level of strain communities: the study-wide comparison identified strain communities of G. vaginalis D, A, and F as associated with BV recurrence and resolution, respectively (FIG. 10C). Other taxa associated with recurrence include Sneathia amnii, Fannyhessea massiliense, and S. vaginalis.

(iii) Microbial mechanisms associated with BV recurrence. Leading hypotheses about the recurrence of BV include metronidazole resistance through ferredoxin/ferredoxin-NADP reductase (FNR) and nitroimidazole reductase, the production of polymicrobial biofilms, and sexual partner reinfection [88-92], though the mechanisms of these hypotheses have yet to be ascertained. Metagenomic data were mapped to VIRGO2, an updated version of the vaginal non-redundant gene database [94]. First, there are unique compositions of Gardnerella species in mgCSTs 20-25 and BV was associated with those containing a greater diversity of Gardnerella species [35], though the functional repertoires of these communities remain unevaluated. The metagenomic data presented herein already provides some novel insights: the presence of additional Gardnerella species yields added virulence factors such as vaginolysin, hemolysin, the muralytic enzyme precursor Rpf2, a vancomycin resistance protein, and glycogen debranching enzymes pullulanase and oligo-16-glucosidase [82]. Next, a COG-directed analyses identified “Extracellular Structure” proteins in S. vaginalis, S. amnii, and Dialister associated with recurrence. Specifically, adhesin proteins homologous to those of Burkholderia pseudomallei, an intracellular agent response for melioidosis, were identified [95,96], and the trimeric autotransporter YadA, a fibronectin-binding adhesin required for epithelial cell invasion by enteropathogenic Yersinia enterocolitica [97]. Hypotheses of the role of biofilms in BV exist [90], but critical evidence of such bacterial adherence in the vagina is lacking.

Further Studies on Bacterial Vaginosis Recurrence (rBV) and Persistence.

Rationale & Hypotheses. Because Clv 19 and Gv 22 were associated with BV recurrence and it is hypothesized this is related to the persistence of specific species or strains in these communities (for example through biofilm formation), this study will address a significant knowledge gap by testing the following hypotheses.
H1: Individuals with Clv 19 or Gv 22 at the index event will be more likely to have persistent BV over 6 months.
H2: Individuals with Clv 19 or Gv 22 at the index event will have fewer new persistent BV infections (defined as a change in mgCST since the last BV event) than other mgCSTs amongst individuals. Furthermore, it is hypothesized that new infections will be more prevalent in those with recurrent versus with persistent BV.
H3: Individuals with Clv 19 or Gv 22 at the index event will have a greater proportion of shared strains between the index and persistent or recurrent BV events than those with any other mgCST.

Study design. To test these hypotheses, metagenomic sequencing data will be generated from LSVF cervicovaginal lavages from the 3- and 6-month follow-up visits of participants that experienced BV recurrence (n=530 samples, the index BV event data are already generated) as well as the index, 3- and 6-month follow-up visits from 196 participants that experience BV persistence (n=588 samples).

Description of the study cohort. This aims seeks to utilize archived samples and data originally collected in the NIH's Longitudinal Study of Vaginal Flora (LSVF, Z01-HD002535) in which 3,620 reproductive-age women were followed for 12 months and assessed quarterly with clinical examinations between August 1999 and February 2002, yielding 13,591 clinical visits. At each visit, participants underwent a pelvic exam and were surveyed on symptoms, demographics and behaviors. For the exam, the clinician placed a speculum, unlubricated or lubricated with water, in the vagina. The quality and consistency of vaginal discharge was described, if present. A vaginal swab was touched to a ColorpHast stick and pH was read. A traditional wet mount (microscopy) was performed to assess for clue cells and whiff test as per Amsel's clinical criteria for the diagnosis of BV. Finally, cervicovaginal lavage (CVL) was collected, aliquoted and stored for further testing at −80° C. Patients with asymptomatic BV are defined as those who did not report vaginal symptoms on direct questioning, but met at least 3 out of 4 Amsel's criteria based on clinician exam (thin, homogenous vaginal discharge, a vaginal pH >4.5, clue cells >20% of epithelial cells and/or a positive whiff test). Patients with symptomatic BV met at least 3 out of 4 Amsel's criteria and reported vaginal symptoms (discharge, vaginal irritation, itching, burning, foul odor or other) when questioned and were treated with standard of care (metronidazole or clindamycin). STIs were tested for at each visit as follows: N. gonorrhea by culture, C. trachomatis by LCR, and T. vaginalis by wet mount. STIs will be controlled for in analyses. All participants were HIV-negative at enrollment. At each visit, participants underwent a detailed interview with a female interviewer.

Metagenomic Sequencing. Accounting for the available data from the preliminary study (index BV event in BV recurrence, from NIH K01), 1,118 additional samples will require metagenomic sequencing. DNA will be extracted using a validated procedure for CVL specimens (˜700 μl) that consistently provides 10-30 μg of high-quality DNA adequate for metagenomic analyses. The aim is to obtain at least 40 million high quality reads for each sample to ensure cost-effective, sufficient coverage for mgCST classification and genome assemblies. Metagenomic libraries will be sequenced on a NovaSeq 6000 platform on an S4 Flowcell at 60 samples per Flowcell.

Sequence processing. Human reads will first be removed from raw sequencing reads using BMTagger, with the GRCh38 human genome as the reference. The remaining non-human metagenomic reads will then be quality filtered using fastp (v.0.21) to remove polyG tails, reads with a minimal length of at least 75 bp, and low-quality reads (—g—1 75—3—W 4—M 20). After quality filtering, the remaining reads will be mapped to the vaginal non-redundant gene database, VIRGO, and mgCSTs assigned with the classifier. Quality-filtered reads will also be assembled into contigs using metaSPAdes. Paired-end reads, as well as unpaired reads, will be passed to the assembler, which will be run with a k-mer range of 21, 33, and 55. The resulting contigs will then be binned using three different tools: MetaBAT, MaxBin, and MetaDecoder. For MetaBAT and MetaDecoder, contigs will be first indexed and reads aligned to the contigs with Bowtie2 (bowtie2-build, bowtie2, respectively). With SAMtools, the alignment results will be output in SAM format, filtered to include only correctly paired reads (samtools view —f 0x2), converted to sorted BAM files (samtools sort), and indexed (samtools index). Next, binning will be performed using MetaBAT, with a maximum edge threshold of 1000 (—maxEdges 1000). Similarly, the indexed and aligned contigs will be the input for MetaDecoder. Contig coverage will be calculated from the aligned SAM files using MetaDecoder's coverage tool, and single-copy marker genes will be mapped to the contigs using the seed function. Finally, contigs will be clustered into bins using metadecoder cluster, with a minimum contig length of 1,000 base pairs. MaxBin will be used to bin contigs longer than 1,000 base pairs (-min_contig_length 1000). The bins generated by MetaBAT, MaxBin, and MetaDecoder will be refined using metaWRAP. Binning refinement will be conducted by setting the minimum completion threshold to 70% (-c 70) and a maximum contamination threshold of 5% (-x 5). The quality of the refined bins will be assessed using CheckM, and taxonomic classification will be performed on the final set of bins using GTDB-Tk (v.2.0.0).

Statistical Analyses. H1: Identifying mgCSTs associated with BV persistence. Applying the same methods used to identify mgCSTs associated with recurrent BV (FIG. 9A, NIH K01, PI Holm), which mgCSTs at the index BV event are related to persistent BV (n=196) over 6 months will be determined compared to BV resolution (n=214, data already generated) (FIG. 9B). The samples for this study are from women experiencing asymptomatic (85% of Amsel-BV positive participants) or symptomatic Amsel-BV and thereby receive treatment. Thus, this variable will be used as a stratum for the associative model. Preliminary analyses using existing metagenomic data from the index BV events prior to BV persistence support the notion that Gv 22 is associated with BV persistence compared to BV resolution (Table 3).

TABLE 3
Vaginal metagenomic community state types (mgCST) differ
in samples prior to persistent Amsel-BV (cases).

| | | No BV Treatment | | | | Received BV Treatment | | | |
|---|---|---|---|---|---|---|---|---|---|
| | | BV | | BV | | BV | | BV | |
| | | Resolves | | Persists | | Resolves | | Persists | |
| | | (n = 175) | | (n = 14) | | (n = 39) | | (n = 5) | |
| Dominant Bacteria | mgCST | n | % | n | % | n | % | n | % |
| Lactobacillus crispatus/gasseri/jensenii | D-LA | 12 | 6.9 | 0 | 0 | 0 | 0 | 0 | 0 |
| Lactobacillus iners | 10 | 5 | 2.9 | 0 | 0 | 1 | 2.6 | 0 | 0 |
| Lactobacillus iners | 11 | 11 | 6.3 | 0 | 0 | 3 | 7.7 | 0 | 0 |
| Lactobacillus iners | 12 | 14 | 8 | 0 | 0 | 2 | 5.1 | 0 | 0 |
| Lactobacillus iners | 13 | 4 | 2.3 | 0 | 0 | 1 | 2.6 | 0 | 0 |
| Lactobacillus iners | 15 | 3 | 1.7 | 0 | 0 | 0 | 0 | 0 | 0 |
| “Ca. Lachnocurva vaginae” | 17 | 14 | 8 | 1 | 7.1 | 4 | 10.3 | 0 | 0 |
| “Ca. Lachnocurva vaginae” | 18 | 6 | 3.4 | 3 | 21.4 | 0 | 0 | 0 | 0 |
| “Ca. Lachnocurva vaginae” | 19 | 2 | 1.1 | 0 | 0 | 1 | 2.6 | 0 | 0 |
| Gardnerella | 20 | 39 | 22.3 | 3 | 21.4 | 6 | 15.4 | 0 | 0 |
| Gardnerella | 22 | 6 | 3.4 | 4 | 28.6 | 2 | 5.1 | 2 | 40 |
| Gardnerella | 23 | 19 | 10.9 | 1 | 7.1 | 6 | 15.4 | 1 | 20 |
| Gardnerella | 24 | 28 | 16 | 2 | 14.3 | 8 | 20.5 | 1 | 20 |
| Gardnerella | 25 | 7 | 4 | 0 | 0 | 0 | 0 | 0 | 0 |
| Gardnerella | 26 | 2 | 1.1 | 0 | 0 | 4 | 10.3 | 0 | 0 |
| Other | 27 | 2 | 1.1 | 0 | 0 | 1 | 2.6 | 1 | 20 |

H2: Determining the rates of new BV infections among recurrent and persistent BV. Among participants that experienced either recurrent or persistent BV (FIG. 9C, see comparisons indicated by “X” and “Y”), the number of new BV infections experienced over time (defined as a change in mgCST since the prior BV event) will be determined to determine the prevalence of new infections among those with either persistent or recurrent BV. Within these groups, it will be determined if individuals with either Clv 19 or Gv 22 at the index BV event have fewer new BV infections compared to those with any other mgCST.

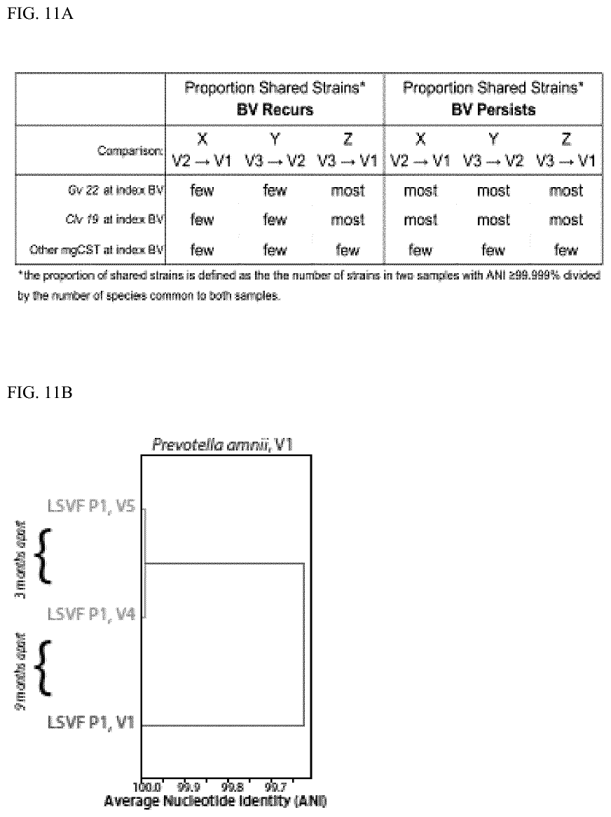

H3: Quantifying the proportions of strains shared in recurrent and persistent BV. To better understand the temporal dynamics of the microbiome during persistent and recurrent BV, the degree of strain sharing between BV events from individuals will be quantified with recurrent and persistent BV quantified using InStrain (FIG. 9C). For each participant, metagenomic reads from the 3-month (V2) and 6-month (V3) visits will be mapped to the high-quality bins (aka, metagenome assembled genomes or MAGs) of the prior visit. The reads from the 6-month visit (V3) will also be mapped to the MAGs of the index event (V1). For each individual, the strain-sharing rate it will be determined, calculated as the number of strains in two samples with ANI≥99.999% (i.e. the same) divided by the number of species common to both samples (i.e. if both samples have a MAG from the same species). Within individuals with persistent or recurrent BV, the strain-sharing rates will be compared between individuals with either Clv 19, Gv 22, or any other mgCST at the index event (FIG. 11A). A preliminary analysis of existing data from an LSVF participant demonstrated identical strains of P. amnii between two time points (3-months apart) which were distinct from the strain from the first visit at which the participant reported recently having had a new sex partner (FIG. 11B).

### The Vaginal Microbiome is Associated With Risk of Incident STIs.

While CSTs, based on 16S rRNA gene amplicons, provide insight into the species composition of the vaginal microbiota, metagenomic CSTs (mgCSTs), based on whole genome shotgun sequencing, offer additional information on the functional potential of the vaginal microbiome.

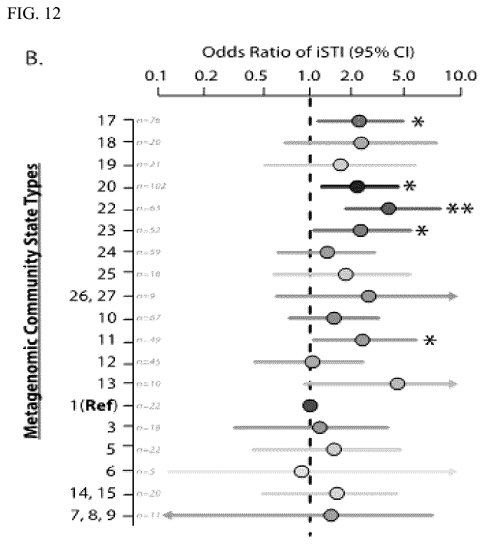

In a study, MgCSTs were assigned in a subset of a cohort (n=708 samples, 1:1 case to matched control). In comparison to a reference L. crispatus-dominated mgCST 1, G. vaginalis-dominated mgCST 22 presented 3.5-fold higher odds of STI acquisition (aOR: 3.53, 95% CI: 1.8-7.9, p=0.001, FIG. 12). Other G. vaginalis predominated mgCSTs, namely 20 and 23, were associated with twofold higher odds (aOR_{mgCST20}: 2.06, 95% CI: 1.2-4.2, aOR_{mgCST23}: 2.39, 95% CI: 1.1-5.4), while G. vaginalis mgCSTs 24 and 25 did not statistically significantly differ from the reference mgCST 1. mgCST 22 was further investigated given its strong link with STI acquisition and it was observed that this mgCST is characterized by genes encoding enzymes involved in glycogen, starch, and mucin degradation. Additionally, mgCST 22 contains genetic markers for epithelial adhesion, such as membrane-bound pili found in Gardnerella sp 11 and G. sp 13, which may serve as pertinent biomarkers for STI risk.

### Discussion

Recent findings that motivated development of mgCST classification included studies showing multiple strains of the same species are commonly observed in the vaginal microbiome [29], and that samples can be clustered into metagenomic subspecies (mgSs) defined by unique strain combinations represented by species-specific gene sets, and thus unique sets of functions. These critical observations led to the conceptualization of a vaginal microbiomes classification based on their mgSs compositions and abundance, and thus defined by both species' composition and functions, i.e., metagenomic community state types (MgCSTs). MgCSTs describe vaginal microbiomes through a new lens, one that includes both compositional and functional dimensions.

L. iners-predominated vaginal microbiota have been associated with increased risks of experiencing bacterial vaginosis (BV) [39, 40]. Longitudinal observational prospective studies support this conclusion and present several critical findings: 1) L. iners is often detected at low to medium abundances during episodes of BV, and L. iners commonly dominate the vaginal microbiota after metronidazole treatment for BV and, 2) L. iners predominated vaginal microbiota are more prevalent prior to incidence of BV [41, 42]. The frequency of L. iners predominated vaginal microbiota observed was high in Black and Hispanic women (31.4% and 36.1%, respectively), both of whom experience a disproportionate prevalence of BV in the US, with reported rates of 33.2% and 30.7%, respectively (compared to 22.7% and 11.1% in White and Asian women) [43]. Interestingly, L. iners predominated vaginal microbiota were even more frequent in North American Asian women in this study, as was shown previously by Ravel et al. [1].

MgCST classification provides insight into this contradiction to prevailing dogma regarding L. iners and increased risk of BV. L. iners mgSs 4 was associated with Amsel-BV, while L. iners mgSs 3 (predominates mgCST 12) was significantly associated with negative BV diagnoses and was most frequently observed in Asian women. This is the first evidence of genetically distinct combinations of L. iners strains (mgSs) in healthy versus BV-like states. This critical finding points to the possibility of beneficial L. iners-dominated microbiomes that had not been evidenced previously.

The analyses presented herein also identified a specific set of L. iners genes associated with positive Amsel-BV diagnoses. Macklaim et al. 2018 reported marked differences in L. iners gene expression between two control patients versus two diagnosed with BV, including increased CRISPR-associated proteins gene expression in BV samples [44]. However, the mgSs analysis of L. iners presented herein indicates that it is not simply alterations in gene expression of a common gene pool that differentiates BV from non-BV microbiomes, but L. iners mgSs that also differ. Microbiomes from women with Amsel-BV diagnoses were enriched for host immune response evasion and host-colonization functions by L. iners. For example, serine/threonine-protein kinases (STPKs) contribute to resistance from phagocytosis by macrophage, invasion of host cells including epithelia and keratinocytes, antibiotic resistance, disruption of the NF-κB signaling pathway, and mucin binding [45]. Bacteria attached to host cells (clue cells) is a hallmark of high Nugent scores (a bacterial morphology-based definition of bacterial vaginosis) and a criterion in Amsel-BV diagnoses [21, 25]. L. iners can appear as Gram-variable cocci (like G. vaginalis) or rods [46, 47], and the data presented herein suggest that certain strains of L. iners (specifically those containing gene cluster 6) may adhere to epithelial cells, contributing to the appearance of clue cells. In addition, epithelial cell adherence could make certain L. iners strains more difficult to displace in the vaginal environment and contribute to the common observation of L. iners following antibiotic treatment [48]. Interestingly, just like L. iners mgSs 4, mgSs of “Ca. Lachnocurva vaginae” were strongly associated with Amsel-BV and were also not found in the vaginal microbiomes of Asian women in this study. Along with the observation that Asian women in this study were more likely to have L. iners mgSs 3 than any other L. iners mgSs and were less burdened with Amsel-BV, it is hypothesized that selective pressures by the host environment may result in niche specialization by vaginal bacteria. Sources of selective pressure could relate to host-provided nutrient availability (e.g., mucus glycan composition), the host innate and adaptive immune system, the circulation of other species' mgSs in a population, or any such combination.

Several distinct mgCSTs associate strongly with Amsel-BV. Critically, these data support the need for an improved definition of BV and the importance of a personalized approach to treatment. “Ca. Lachnocurva vaginae” predominated mgCSTs were strongly associated with asymptomatic Amsel-BV and contained more high Nugent scores than other mgCSTs. Conversely, intermediate Nugent scores were most prevalent in G. vaginalis predominated mgCSTs, and only three of these six mgCSTs were associated with Amsel-BV, which suggests that not all G. vaginalis-dominated microbiomes are related to Amsel-BV. G. vaginalis contains vast genomic diversity, supporting a split into different genomospecies [38, 49, 50]. Because different genomospecies can co-exist, the data presented herein show that G. vaginalis predominated mgSs represent unique combinations of genomospecies and strains of these genomospecies. MgCSTs 20-22 contain high Gardnerella genomospecies diversity and were associated with positive Amsel-BV diagnoses in studies using qPCR or transcriptomic data to define Gardnerella species [49, 51-53]. The data presented herein corroborate these reports and further indicate in mgCSTs with higher numbers of Gardnerella genomospecies that there are more gene variants coding for virulence factors like cholesterol-dependent pore-forming cytotoxin vaginolysin and neuraminidase sialidase present, thus expanding functional redundancy of these enzymes and potentially contributing to the association with positive Amsel-BV diagnoses [54-56]. However, mgCST 24 which is comprised of G. vaginalis mgSs 4 (G. vaginalis and G. swidsinkii), has relatively lower Gardnerella genomospecies diversity and was highly associated with Amsel-BV and symptomatic Amsel-BV. Together these data suggest that enumeration and classification of Gardnerella genomospecies may prove to be an important diagnostic of different “types” of Amsel-BV which could inform treatment options. For example, it is possible that harboring more Gardnerella genomospecies may predict BV recurrence following metronidazole treatment, suggesting the need for a different approach to treatment. Alternatively, some Gardnerella genomospecies may be important and novel targets of therapy.

In the clinic, antibiotic treatment is recommended for BV diagnosis (generally a point-of-care test) only when the patient reports symptoms, which is estimated to occur in fewer than half of women with BV [24, 57, 58]. In research settings, both symptomatic and asymptomatic Amsel-BV can be evaluated. Indeed, in the observational research studies included in this analysis where Amsel criteria were evaluated along with whether participants reported symptoms or not, symptomatic Amsel-BV accounted for only 12% of Amsel-BV cases and 30% of these were in mgCST 24 (dominated primarily by Gardnerella swidsinkii and G. vaginalis). It is hypothesized that the high prevalence of BV recurrence post-treatment may be due to the heterogeneity in the genetic make-up of the microbiota associated with BV as revealed by mgCSTs. MgCSTs reduce this heterogeneity resulting in more precise estimates of risk. Furthermore, these findings highlight the potential importance of developing specialized treatments that target “types” of BV.

The mgCST framework can also be used to identify vaginal microbiomes that are associated with positive health outcomes. For example, mgCSTs predominated by different L. crispatus mgSs varied in their association with low Nugent scores, the number of L. crispatus strains present, and the longitudinal stability of communities. The vaginal microbiome can be dynamic [59-61]. Shifts from Lactobacillus to non-Lactobacillus predominated microbiota can increase the risk of infection following exposure to a pathogen. The present study identified L. crispatus mgCSTs with variable stability, suggesting that not all L. crispatus predominated microbiomes are functionally similar and may be differently permissive to infection. Those found to be associated with higher stability may reduce the window of opportunity for pathogens to invade. Microbiome stability may be related to both the diversity of other non-Lactobacillus members of the microbiome and/or the number of L. crispatus strains present. In any case, the present study shows that there is a range of protective abilities even among L. crispatus predominated communities. This information could be critical in selecting and assembling strains of L. crispatus to design novel live biotherapeutics products aimed to restore an optimal vaginal microenvironment.

To aid in further exploration, a validated classifier for both mgSs and mgCSTs is provided at github.com/ravel-lab/mgCST-classifier/blob/main/README.md.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
1. Ravel J, Gajer P, Abdo Z, Schneider GM, Koenig SSK, McCulle SL, et al.: Vaginal microbiome of reproductive-age women. In: Proc Natl Acad Sci USA. vol. 108 Suppl 1; 2011: 4680-7.
2. O'Hanlon DE, Come RA, Moench TR. Vaginal pH measured in vivo: lactobacilli determine pH and lactic acid concentration. Bmc Microbiol. 2019;19(1):13; doi: 10.1186/s12866-019-1388-8.
3. Gong Z, Luna Y, Yu P, Fan H. Lactobacilli inactivate Chlamydia trachomatis through lactic acid but not H2O2. PLoS One. 2014;9(9):e107758; doi: 10.1371/journal.pone.0107758.
4. Witkin SS, Mendes-Soares H, Linhares IM, Jayaram A, Ledger WJ, Forney LJ. Influence of vaginal bacteria and D- and L-lactic acid isomers on vaginal extracellular matrix metalloproteinase inducer: implications for protection against upper genital tract infections. mBio. 2013;4(4); doi: 10.1128/mBio.00460-13.
5. Ravel J, Brotman RM. Translating the vaginal microbiome: gaps and challenges. Genome Med. 2016;8(1):35; doi: 10.1186/s13073-016-0291-2.
6. Amabebe E, Anumba DOC. The Vaginal Microenvironment: The Physiologic Role of Lactobacilli. Front Med (Lausanne). 2018;5:181; doi: 10.3389/fmed.2018.00181.
7. Ma B, Forney LJ, Ravel J. Vaginal microbiome: rethinking health and disease. Annu Rev Microbiol. 2012;66:371-89; doi: 10.1146/annurev-micro-092611-150157.
8. Boskey ER, Cone RA, Whaley KJ, Moench TR: Origins of vaginal acidity: high D/L lactate ratio is consistent with bacteria being the primary source. In: Hum Reprod. vol. 16: Oxford University Press; 2001:1809-13.
9. Nunn KL, Wang YY, Harit D, Humphrys MS, Ma B, Cone R, et al. Enhanced Trapping of HIV-1 by Human Cervicovaginal Mucus Is Associated with Lactobacillus crispatus-Dominant Microbiota. mBio. 2015;6(5):e01084-15; doi: 10.1128/mBio.01084-15.
10. Edwards VL, Smith SB, McComb EJ, Tamarelle J, Ma B, Humphrys MS, et al. The Cervicovaginal Microbiota-Host Interaction Modulates Chlamydia trachomatis Infection. mBio. 2019;10(4); doi: 10.1128/mBio.01548-19.
11. France MT, Ma B, Gajer P, Brown S, Humphrys MS, Holm JB, et al. VALENCIA: a nearest centroid classification method for vaginal microbial communities based on composition. Microbiome. 2020;8(1):166; doi: 10.1186/s40168-020-00934-6.
12. Brotman RM, Bradford LL, Conrad M, Gajer P, Ault K, Peralta L, et al. Association between Trichomonas vaginalis and vaginal bacterial community composition among reproductive-age women. Sexually transmitted diseases. 2012;39(10):807-12; doi: 10.1097/OLQ.0b013e3182631c79.
13. Mehta SD, Donovan B, Weber KM, Cohen M, Ravel J, Gajer P, et al. The vaginal microbiota over an 8- to 10-year period in a cohort of HIV-infected and HIV-uninfected women. PLoS One. 2015;10(2):e0116894; doi: 10.1371/journal.pone.0116894.
14. Dunlop AL, Satten GA, Hu YJ, Knight AK, Hill CC, Wright ML, et al. Vaginal Microbiome Composition in Early Pregnancy and Risk of Spontaneous Preterm and Early Term Birth Among African American Women. Front Cell Infect Microbiol. 2021;11:641005; doi: 10.3389/fcimb.2021.641005.
15. Price JT, Vwalika B, Hobbs M, Nelson JAE, Stringer EM, Zou F, et al. Highly diverse anaerobe-predominant vaginal microbiota among HIV-infected pregnant women in Zambia. PLoS One. 2019;14(10):e0223128; doi: 10.1371/journal.pone.0223128.
16. Gosmann C, Anahtar MN, Handley SA, Farcasanu M, Abu-Ali G, Bowman BA, et al. Lactobacillus-Deficient Cervicovaginal Bacterial Communities Are Associated with Increased HIV Acquisition in Young South African Women. Immunity. 2017;46(1):29-37; doi: 10.1016/j.immuni.2016.12.013.
17. Atashili J, Poole C, Ndumbe PM, Adimora AA, Smith JS. Bacterial vaginosis and HIV acquisition: a meta-analysis of published studies. AIDS. 2008;22(12):1493-501; doi: 10.1097/QAD.0b013e3283021a37.
18. Elovitz MA, Gajer P, Riis V, Brown AG, Humphrys MS, Holm JB, et al. Cervicovaginal microbiota and local immune response modulate the risk of spontaneous preterm delivery. Nat Commun. 2019;10(1):1305; doi: 10.1038/s41467-019-09285-9.
19. Borgdorff H, Tsivtsivadze E, Verhelst R, Marzorati M, Jurriaans S, Ndayisaba GF, et al. Lactobacillus-dominated cervicovaginal microbiota associated with reduced HIV/STI prevalence and genital HIV viral load in African women. ISME J. 2014;8(9):1781-93; doi: 10.1038/ismej.2014.26.
20. Tamarelle J, Thiebaut ACM, de Barbeyrac B, Bebear C, Ravel J, Delarocque-Astagneau E. The vaginal microbiota and its association with human papillomavirus, Chlamydia trachomatis, Neisseria gonorrhoeae and Mycoplasma genitalium infections: a systematic review and meta-analysis. Clin Microbiol Infect. 2019;25(1):35-47; doi: 10.1016/j.cmi.2018.04.019.
21. Amsel R, Totten PA, Spiegel CA, Chen KC, Eschenbach D, Holmes KK. Nonspecific vaginitis: diagnostic criteria and microbial and epidemiologic associations. The American journal of medicine. 1983;74(1):14-22.
22. Scharbo-Dehaan M, Anderson DG. The CDC 2002 guidelines for the treatment of sexually transmitted diseases: implications for women's health care. J Midwifery Womens Health. 2003;48(2):96-104; doi: 10.1016/s1526-9523(02)00416-6.
23. Bilardi JE, Walker S, Temple-Smith M, McNair R, Mooney-Somers J, Bellhouse C, et al. The burden of bacterial vaginosis: women's experience of the physical, emotional, sexual and social impact of living with recurrent bacterial vaginosis. PLoS One. 2013;8(9):e74378; doi: 10.1371/journal.pone.0074378.
24. Klebanoff MA, Schwebke JR, Zhang J, Nansel TR, Yu KF, Andrews WW. Vulvovaginal symptoms in women with bacterial vaginosis. Obstet Gynecol. 2004;104(2):267-72; doi: 10.1097/01.AOG.0000134783.98382.b0.
25. Nugent RP, Krohn MA, Hillier SL. Reliability of diagnosing bacterial vaginosis is improved by a standardized method of gram stain interpretation. Journal of clinical microbiology. 1991;29(2):297-301; doi: 10.1128/jcm.29.2.297-301.1991.
26. Mckinnon LR, Achilles SL, Bradshaw CS, Burgener A, Crucitti T, Fredricks DN, et al. The Evolving Facets of Bacterial Vaginosis: Implications for HIV Transmission. AIDS Res Hum Retroviruses. 2019;35(3):219-28; doi: 10.1089/AID.2018.0304.
27. Sela U, Euler CW, Correa da Rosa J, Fischetti VA. Strains of bacterial species induce a greatly varied acute adaptive immune response: The contribution of the accessory genome. PLoS Pathog. 2018;14(1):e1006726; doi: 10.1371/journal.ppat.1006726.
28. Douillard FP, Ribbera A, Kant R, Pietila TE, Jarvinen HM, Messing M, et al. Comparative genomic and functional analysis of 100 Lactobacillus rhamnosus strains and their comparison with strain GG. PLoS Genet. 2013;9(8):e1003683; doi: 10.1371/journal.pgen.1003683.
29. Ma B, France MT, Crabtree J, Holm JB, Humphrys MS, Brotman RM, et al. A comprehensive non-redundant gene catalog reveals extensive within-community intraspecies diversity in the human vagina. Nat Commun. 2020;11(1):940; doi: 10.1038/s41467-020-14677-3.
30. Tortelli BA, Lewis AL, Fay JC. The structure and diversity of strain-level variation in vaginal bacteria. Microb Genom. 2021; 7(3); doi: 10.1099/mgen.0.000543.
31. O'Hanlon DE, Moench TR, Cone RA. Vaginal pH and microbicidal lactic acid when lactobacilli dominate the microbiota. PLoS One. 2013;8(11):e80074; doi: 10.1371/journal.pone.0080074.
32. Tachedjian G, Aldunate M, Bradshaw CS, Cone RA. The role of lactic acid production by probiotic Lactobacillus species in vaginal health. Res Microbiol. 2017;168(9-10):782-92; doi: 10.1016/j.resmic.2017.04.001.
33. Kochhar S, Hottinger H, Chuard N, Taylor PG, Atkinson T, Scawen MD, et al. Cloning and overexpression of Lactobacillus helveticus D-lactate dehydrogenase gene in Escherichia coli. Eur J Biochem. 1992;208(3):799-805; doi: 10.1111/j.1432-1033.1992.tb17250.x.
34. Petrova MI, Reid G, Vaneechoutte M, Lebeer S. Lactobacillus iners: Friend or Foe? Trends Microbiol. 2017;25(3):182-91; doi: 10.1016/j.tim.2016.11.007.
35. Whelan F, Lafita A, Gilburt J, Degut C, Griffiths SC, Jenkins HT, et al. Periscope Proteins are variable-length regulators of bacterial cell surface interactions. Proc Natl Acad Sci USA. 2021;118(23); doi: 10.1073/pnas.2101349118.
36. Gaillard JL, Berche P, Frehel C, Gouin E, Cossart P. Entry of L. monocytogenes into cells is mediated by internalin, a repeat protein reminiscent of surface antigens from gram-positive cocci. Cell. 1991;65(7):1127-41; doi: 10.1016/0092-8674(91)90009-n.
37. Rampersaud R, Planet PJ, Randis TM, Kulkarni R, Aguilar JL, Lehrer RI, et al. Inerolysin, a cholesterol-dependent cytolysin produced by Lactobacillus iners. J Bacteriol. 2011;193(5):1034-41; doi: 10.1128/JB.00694-10.
38. Vaneechoutte M, Guschin A, Van Simaey L, Gansemans Y, Van Nieuwerburgh F, Cools P. Emended description of Gardnerella vaginalis and description of Gardnerella leopoldii sp. nov., Gardnerella piotii sp. nov. and Gardnerella swidsinskii sp. nov., with delineation of 13 genomic species within the genus Gardnerella. Int J Syst Evol Microbiol. 2019;69(3):679-87; doi: 10.1099/ijsem.0.003200.
39. Muzny CA, Blanchard E, Taylor CM, Aaron KJ, Talluri R, Griswold ME, et al. Identification of Key Bacteria Involved in the Induction of Incident Bacterial Vaginosis: A Prospective Study. J Infect Dis. 2018;218(6):966-78; doi: 10.1093/infdis/jiy243.
40. Verstraelen H, Verhelst R, Claeys G, De Backer E, Temmerman M, Vaneechoutte M. Longitudinal analysis of the vaginal microflora in pregnancy suggests that L. crispatus promotes the stability of the normal vaginal microflora and that L. gasseri and/or L. iners are more conducive to the occurrence of abnormal vaginal microflora. BMC Microbiol. 2009;9:116; doi: 10.1186/1471-2180-9-116.
41. Ravel J, Brotman RM, Gajer P, Ma B, Nandy M, Fadrosh DW, et al. Daily temporal dynamics of vaginal microbiota before, during and after episodes of bacterial vaginosis. Microbiome. 2013;1(1):29; doi: 10.1186/2049-2618-1-29.
42. Ferris MJ, Norori J, Zozaya-Hinchliffe M, Martin DH: Cultivation-Independent Analysis of Changes in Bacterial Vaginosis Flora Following Metronidazole Treatment. In: Journal of Clinical Microbiology. vol. 45; 2007: 1016-8.
43. Peebles K, Velloza J, Balkus JE, McClelland RS, Barnabas RV. High Global Burden and Costs of Bacterial Vaginosis: A Systematic Review and Meta-Analysis. Sex Transm Dis. 2019;46(5):304-11; doi: 10.1097/OLQ.0000000000000972.
44. Macklaim JM, Fernandes AD, Di Bella JM, Hammond J-A, Reid G, Gloor GB: Comparative meta-RNA-seq of the vaginal microbiota and differential expression by Lactobacillus iners in health and dysbiosis. In: Microbiome. vol. 1; 2013: 12.
45. Canova MJ, Molle V. Bacterial serine/threonine protein kinases in host-pathogen interactions. J Biol Chem. 2014;289(14):9473-9; doi: 10.1074/jbc.R113.529917.
46. Kim H, Kim T, Kang J, Kim Y, Kim H. Is Lactobacillus Gram-Positive? A Case Study of Lactobacillus iners. Microorganisms. 2020;8(7); doi: 10.3390/microorganisms8070969.
47. Holm JB, Carter KA, Ravel J, Brotman RM. Lactobacillus iners and Genital Health: Molecular Clues to an Enigmatic Vaginal Species. Current Infectious Disease Reports. 2023;25(4):67-75; doi: 10.1007/s11908-023-00798-5.
48. Tamarelle J, Ma B, Gajer P, Humphrys MS, Terplan M, Mark KS, et al. Nonoptimal Vaginal Microbiota After Azithromycin Treatment for Chlamydia trachomatis Infection. J Infect Dis. 2020;221(4):627-35; doi: 10.1093/infdis/jiz499.
49. Potter RF, Burnham CD, Dantas G. In Silico Analysis of Gardnerella Genomospecies Detected in the Setting of Bacterial Vaginosis. Clin Chem. 2019;65(11):1375-87; doi: 10.1373/clinchem.2019.305474.
50. Ksiezarek M, Ugarcina-Perovic S, Rocha J, Grosso F, Peixe L. Long-term stability of the urogenital microbiota of asymptomatic European women. Bmc Microbiol. 2021;21(1):64; doi: 10.1186/s12866-021-02123-3.
51. Turner E, Sobel JD, Akins RA. Prognosis of recurrent bacterial vaginosis based on longitudinal changes in abundance of Lactobacillus and specific species of Gardnerella. PLoS One. 2021;16(8):e0256445; doi: 10.1371/journal.pone.0256445.
52. Zozaya-Hinchliffe M, Lillis R, Martin DH, Ferris MJ: Quantitative PCR Assessments of Bacterial Species in Women with and without Bacterial Vaginosis. In: Journal of Clinical Microbiology. vol. 48; 2010: 1812-9.
53. Janulaitiene M, Paliulyte V, Grinceviciene S, Zakareviciene J, Vladisauskiene A, Marcinkute A, et al. Prevalence and distribution of Gardnerella vaginalis subgroups in women with and without bacterial vaginosis. BMC Infect Dis. 2017;17(1):394; doi: 10.1186/s12879-017-2501-y.
54. Gelber SE, Aguilar JL, Lewis KL, Ratner AJ. Functional and phylogenetic characterization of Vaginolysin, the human-specific cytolysin from Gardnerella vaginalis. J Bacteriol. 2008;190(11):3896-903; doi: 10.1128/JB.01965-07.
55. Pleckaityte M, Janulaitiene M, Lasickiene R, Zvirbliene A. Genetic and biochemical diversity of Gardnerella vaginalis strains isolated from women with bacterial vaginosis. FEMS Immunol Med Microbiol. 2012;65(1):69-77; doi: 10.1111/j.1574-695X.2012.00940.x.
56. Yeoman CJ, Yildirim S, Thomas SM, Durkin AS, Torralba M, Sutton G, et al. Comparative genomics of Gardnerella vaginalis strains reveals substantial differences in metabolic and virulence potential. PLoS One. 2010;5(8):e12411; doi: 10.1371/journal.pone.0012411.
57. Koumans EH, Sternberg M, Bruce C, McQuillan G, Kendrick J, Sutton M, et al. The prevalence of bacterial vaginosis in the United States, 2001-2004; associations with symptoms, sexual behaviors, and reproductive health. Sex Transm Dis. 2007;34(11):864-9; doi: 10.1097/OLQ.0b013e318074e565.
58. Workowski KA, Bachmann LH, Chan PA, Johnston CM, Muzny CA, Park I, et al. Sexually Transmitted Infections Treatment Guidelines, 2021. MMWR Recomm Rep. 2021;70(4):1-187; doi: 10.15585/mmwr.rr7004a1.
59. Brotman RM, Ravel J, Cone RA, Zenilman JM. Rapid fluctuation of the vaginal microbiota measured by Gram stain analysis. Sex Transm Infect. 2010;86(4):297-302; doi: 10.1136/sti.2009.040592.
60. Gajer P, Brotman RM, Bai G, Sakamoto J, Schütte UME, Zhong X, et al. Temporal Dynamics of the Human Vaginal Microbiota. Sci Transl Med. 2012;4(132):132ra52-ra52.
61. Munoz A, Hayward MR, Bloom SM, Rocafort M, Ngcapu S, Mafunda NA, et al. Modeling the temporal dynamics of cervicovaginal microbiota identifies targets that may promote reproductive health. Microbiome. 2021;9(1):163; doi: 10.1186/s40168-021-01096-9.
62. Li F, Chen C, Wei W, Wang Z, Dai J, Hao L, et al. The metagenome of the female upper reproductive tract. Gigascience. 2018;7(10); doi: 10.1093/gigascience/giy107.
63. Bolger AM, Lohse M, Usadel B. Trimmomatic: a flexible trimmer for Illumina sequence data. Bioinformatics. 2014;30(15):2114-20; doi: 10.1093/bioinformatics/btu170.
64. Parks DH, Chuvochina M, Waite DW, Rinke C, Skarshewski A, Chaumeil PA, et al. A standardized bacterial taxonomy based on genome phylogeny substantially revises the tree of life. Nat Biotechnol. 2018;36(10):996-1004; doi: 10.1038/nbt.4229.
65. Oksanen J, Kindt R, Legendre P, O'Hara B, Stevens MHH, Oksanen MJ, et al. The vegan package. Community ecology package. 2007;10:631-7.
66. Langfelder P, Zhang B, Horvath S. Defining clusters from a hierarchical cluster tree: the Dynamic Tree Cut package for R. Bioinformatics. 2008;24(5):719-20; doi: 10.1093/bioinformatics/btm563.
67. Warnes MGR, Bolker B, Bonebakker L, Gentleman R. Package ‘gplots’. Various R Programming Tools for Plotting Data. 2016.
68. Hennig C. Cluster-wise assessment of cluster stability. Computational Statistics & Data Analysis. 2007;52(1):258-71.
69. Hennig C. Dissolution point and isolation robustness: Robustness criteria for general cluster analysis methods. Journal of Multivariate Analysis. 2008;99(6):1154-76; doi: https://doi.org/10.1016/j.jmva.2007.07.002.
70. Yue JC, Clayton MK. A similarity measure based on species proportions. Communications in Statistics-theory and Methods. 2005;34(11):2123-31.
71. U.B. IHaK. Fast Unified Random Forests for Survival, Regression, and Classification (RF-SRC), R package. 2021.
72. Feehily C, Crosby D, Walsh CJ, Lawton EM, Higgins S, McAuliffe FM, et al. Shotgun sequencing of the vaginal microbiome reveals both a species and functional potential signature of preterm birth. NPJ Biofilms Microbiomes. 2020;6(1):50; doi: 10.1038/s41522-020-00162-8.
73. Yang Q, Wang Y, Wei X, Zhu J, Wang X, Xie X, et al. The Alterations of Vaginal Microbiome in HPV16 Infection as Identified by Shotgun Metagenomic Sequencing. Front Cell Infect Microbiol. 2020;10:286; doi: 10.3389/fcimb.2020.00286.
74. France MT, Brown SE, Rompalo AM, Brotman RM, Ravel J. Identification of shared bacterial strains in the vaginal microbiota of related and unrelated reproductive-age mothers and daughters using genome-resolved metagenomics. PLoS One. 2022;17(10):e0275908; doi: 10.1371/journal.pone.0275908.
75. Rotmistrovsky K, Agarwala R. BMTagger: Best Match Tagger for removing human reads from metagenomics datasets. 2011.
76. Kopylova E, Noe L, Touzet H. SortMeRNA: fast and accurate filtering of ribosomal RNAs in metatranscriptomic data. Bioinformatics. 2012;28(24):3211-7; doi: 10.1093/bioinformatics/bts611.
77. Chen S, Zhou Y, Chen Y, Gu J. fastp: an ultra-fast all-in-one FASTQ preprocessor. Bioinformatics. 2018;34(17):1884-190; doi: 10.1093/bioinformatics/bty560.
78. Jespers, V. et al. Prevalence and correlates of bacterial vaginosis in different sub-populations of women in sub-Saharan Africa: a cross-sectional study. PLoS one 9, e109670 (2014).
79. Demba, E. et al. Bacterial vaginosis, vaginal flora patterns and vaginal hygiene practices in patients presenting with vaginal discharge syndrome in The Gambia, West Africa. BMC infectious diseases 5, 12 (2005).
80. Torrone, E. A. et al. Prevalence of sexually transmitted infections and bacterial vaginosis among women in sub-Saharan Africa: an individual participant data meta-analysis of 18 HIV prevention studies. PLoS medicine 15, e1002511 (2018).
81. Kenyon, C., Colebunders, R. & Crucitti, T. The global epidemiology of bacterial vaginosis: a systematic review. American journal of obstetrics and gynecology 209, 505-523 (2013).
82. Holm, J. B. et al. Integrating compositional and functional content to describe vaginal microbiomes in health and disease. Microbiome 11, 259 (2023). https://doi.org:10.1186/s40168-023-01692-x.
83. Ness, R. B. et al. Can known risk factors explain racial differences in the occurrence of bacterial vaginosis? J Natl Med Assoc 95, 201-212 (2003).
84. Bradshaw, C. S. et al. High recurrence rates of bacterial vaginosis over the course of 12 months after oral metronidazole therapy and factors associated with recurrence. J Infect Dis 193, 1478-1486 (2006). https://doi.org:10.1086/503780.
85. Xiao, B. et al. Association Analysis on Recurrence of Bacterial Vaginosis Revealed Microbes and Clinical Variables Important for Treatment Outcome. Front Cell Infect Microbiol 9, 189 (2019). https://doi.org:10.3389/fcimb.2019.00189.
86. Nelson, T. M. et al. Cigarette smoking is associated with an altered vaginal tract metabolomic profile. Sci Rep 8, 852 (2018). https://doi.org:10.1038/s41598-017-14943-3.
87. Sobel, J. D., Schmitt, C. & Meriwether, C. Long-Term Follow-Up of Patients with Bacterial Vaginosis Treated with Oral Metronidazole and Topical Clindamycin. The Journal of Infectious Diseases 167, 783-784 (1993). https://doi.org:10.1093/infdis/167.3.783.
88. Gustin, A. T. et al. Recurrent bacterial vaginosis following metronidazole treatment is associated with microbiota richness at diagnosis. Am J Obstet Gynecol 226, 225 e221-225 e215 (2022). https://doi.org:10.1016/j.ajog.2021.09.018.
89. Machado, D., Castro, J., Palmeira-de-Oliveira, A., Martinez-de-Oliveira, J. & Cerca, N. Bacterial Vaginosis Biofilms: Challenges to Current Therapies and Emerging Solutions. Front Microbiol 6, 1528 (2015). https://doi.org:10.3389/fmicb.2015.01528.
90. Muzny, C. A. et al. An Updated Conceptual Model on the Pathogenesis of Bacterial Vaginosis. J Infect Dis 220, 1399-1405 (2019). https://doi.org:10.1093/infdis/jiz342.
91. Sousa, L. G. V., Pereira, S. A. & Cerca, N. Fighting polymicrobial biofilms in bacterial vaginosis. Microb Biotechnol 16, 1423-1437 (2023). https://doi.org:10.1111/1751-7915.14261.
92. Deng, Z. L. et al. Metatranscriptome Analysis of the Vaginal Microbiota Reveals Potential Mechanisms for Protection against Metronidazole in Bacterial Vaginosis. mSphere 3 (2018). https://doi.org:10.1128/mSphereDirect.00262-18.
93. Centers for Disease, C. Sexually transmitted diseases treatment guidelines 1982. MMWR Morb Mortal Wkly Rep 31 Suppl 2, 33S-60S (1982).
94. Ma, B. et al. A comprehensive non-redundant gene catalog reveals extensive within-community intraspecies diversity in the human vagina. Nat Commun 11, 940 (2020). https://doi.org:10.1038/s41467-020-14677-3.
95. Wiersinga, W. J., van der Poll, T., White, N. J., Day, N. P. & Peacock, S. J. Melioidosis: insights into the pathogenicity of Burkholderia pseudomallei. Nat Rev Microbiol 4, 272-282 (2006). https://doi.org:10.1038/nrmicro1385.
96. Brown, N. F., Boddey, J. A., Flegg, C. P. & Beacham, I. R. Adherence of Burkholderia pseudomallei cells to cultured human epithelial cell lines is regulated by growth temperature. Infect Immun 70, 974-980 (2002). https://doi.org:10.1128/IAI.70.2.974-980.2002.
97. Heise, T. & Dersch, P. Identification of a domain in Yersinia virulence factor YadA that is crucial for extracellular matrix-specific cell adhesion and uptake. Proc Natl Acad Sci USA 103, 3375-3380 (2006). https://doi.org:10.1073/pnas.0507749103

What is claimed is:

1. A method of characterizing a vaginal microbiome, comprising:
obtaining a vaginal microbiome sample from a subject,
determining the metagenomic community state type (MgCST) of the sample, and classifying the MgCST as one of MgCSTs 1-27.

2. The method of claim 1, further comprising determining one or more of (a) age of the subject, (b) race of the subject, (c) vaginal pH of the subject, and (d) Gram stain assessment of the subject.

3. The method of claim 1, further comprising determining one or more metagenomic subspecies (mgSs) cluster in the sample.

4. The method of claim 1, wherein the MgCST is determined by a two-step classifier that assigns mgCSTs.

5. The method of claim 4, wherein the classifier uses a vaginal non-redundant gene database (VIRGO).

6. The method of claim 1, wherein the subject is a human.

7. A method of identifying a subject predisposed to develop bacterial vaginosis, comprising:
obtaining a vaginal microbiome sample from a subject, and
determining the metagenomic community state type (MgCST) of the sample,
wherein when the MgCST of the subject is determined to be a MgCST associated with bacterial vaginosis, the subject is identified as a subject predisposed to develop bacterial vaginosis.

8. The method of claim 7, further comprising determining one or more of (a) age of the subject, (b) race of the subject, (c) vaginal pH of the subject, and (d) Gram stain assessment of the subject.

9. The method of claim 7, further comprising determining one or more metagenomic subspecies (mgSs) cluster in the sample.

10. The method of claim 7, wherein the MgCST is determined by a two-step classifier that assigns mgCSTs.

11. The method of claim 10, wherein the classifier uses a vaginal non-redundant gene database (VIRGO).

12. The method of claim 7, wherein the subject is a human.

13. The method of claim 7, wherein a MgCST associated with bacterial vaginosis is one or more of MgCSTs 12 and 17-25.

14. The method of claim 7, wherein a MgCST associated with bacterial vaginosis is one or more of MgCSTs 19 and 22.

15. A method of identifying a subject predisposed to re-develop bacterial vaginosis, comprising determining the metagenomic community state type (MgCST) of a vaginal microbiome of a subject after treatment for bacterial vaginosis and classifying it as one of MgCSTs 1-27, wherein when the MgCST of the subject is classified as one or more of MgCSTs 12 and 17-25, the subject is predisposed to re-develop bacterial vaginosis.

16. The method of claim 15, further comprising determining one or more of (a) age of the subject, (b) race of the subject, (c) vaginal pH of the subject, and (d) Gram stain assessment of the subject.

17. The method of claim 15, further comprising determining one or more metagenomic subspecies (mgSs) cluster in the sample.

18. The method of claim 15, wherein the MgCST is determined by a two-step classifier that assigns mgCSTs.

19. The method of claim 18, wherein the classifier uses a vaginal non-redundant gene database (VIRGO).

20. The method of claim 15, wherein the subject is a human.

21. The method of claim 15, wherein when the MgCST of the subject is classified as one or more of MgCSTs 19 and 22, the subject is predisposed to re-develop bacterial vaginosis.
